(19) United States
(12) Patent Application Publication
(10) Pub. No.: US 2024/0293074 A1
(43) Pub. Date: Sep. 5, 2024

(54) PATIENT SUPPORT APPARATUSES WITH PATIENT MONITORING

(71) Applicant: Stryker Corporation, Kalamazoo, MI (US)

(72) Inventors: Jerald A. Trepanier, Augusta, MI (US); Krishna Sandeep Bhimavarapu, Kalamazoo, MI (US)

(21) Appl. No.: 18/573,155

(22) PCT Filed: Sep. 15, 2022
(86) PCT No.: PCT/US2022/043586

§ 371 Date: Dec. 21, 2023

Related U.S. Application Data
(60) Provisional application No. 63/245,279, filed on Sep. 17, 2021.

(51) Int. Cl. A61B 5/00; A61B 5/11; A61B 90/98; A61G 7/057; G16H 40/67

(52) U.S. Cl. CPC A61B 5/447; A61B 5/1115; A61B 5/1127; A61B 90/98; A61G 7/057; G16H 40/67; A61G 2203/36

(57) ABSTRACT

A patient support apparatus for supporting a patient wirelessly communicates with one or more tags using one or more RF transceivers. A controller onboard the patient support apparatus determines a position of the tag. The controller also receives an identifier from the tag and uses it to determine if the tag is attached to a patient or a device. If attached to a patient, the controller may allow a caregiver to activate a patient movement monitoring function. The movement monitoring function may monitor whether the patient exits the patient support apparatus, whether the patient has been turned, whether the patient is at risk of developing bed sores, and/or other aspects. If the tag is attached to a device, the controller automatically determines whether to associate the device with the patient support apparatus. The controller may also determine the location of the patient support apparatus from the RF transceivers.

### BACKGROUND

The present disclosure relates to patient support apparatuses, such as beds, cots, stretchers, recliners, or the like.

Patient support apparatuses often include a built-in exit detection system that monitors the distribution of the patient's weight on a support surface of the patient support apparatus. If the patient's weight shifts in a manner indicative of an exit, the exit detection system issues an alarm. The patient support apparatus therefore monitors changes in the weight distribution of the patient using one or more weight sensors. Although such weight monitoring provides good information about whether the patient has exited the patient support apparatus or not, it may be desirable to capture additional information about the patient's movement, such as whether the patient has been turned, whether the patient is at risk for developing bed sores, whether the patient is restless, and/or other information regarding the patient's movement or lack of movement.

### SUMMARY

According to the various aspects described herein, the present disclosure is directed to a patient monitoring system that monitors the movement of one or more locations on the patient's body and, in some instances, takes different actions depending upon the particular movement of the monitored location(s) on the patient's body. Such actions may include issuing an exit alert, issuing a notification that a patient has been turned (and/or should be turned), issuing a notification that a patient is restless, issuing a notification that a patient is at risk of developing bed sores, and/or issuing other notifications. The monitoring of the movement of the one or more locations on the patient's body is accomplished through one or more tags that are coupled to the patient's body. The tag(s) are adapted to wirelessly communicate with one or more transceivers built into the patient support apparatus that are adapted to repetitively determine the location of the tag(s) with respect to the patient support apparatus. In some aspects, the transceivers communicate with the tag(s) using ultra-wideband technology. Additional aspects of the present disclosure are described in more detail below.

According to one aspect of the present disclosure, a patient support apparatus is provided that includes a support surface adapted to support a patient, a first transceiver, and a controller. The first transceiver is coupled to a first location on the patient support apparatus and is in communication with the controller. The controller is adapted to use radio frequency (RF) communication between the first transceiver and a first tag to determine a first distance between the first tag and the first transceiver. The first tag is coupled to a patient associated with the patient support apparatus.

According to another aspect, the first transceiver is an ultra-wideband transceiver.

In some aspects, the controller is further adapted to issue an exit alert when the first distance exceeds a threshold.

The controller, in some aspects, is further adapted to repetitively determine the first distance between the first tag and the first transceiver and to record changes in the first distance.

The patient support apparatus, in some aspects, further includes a second transceiver adapted to communicate with server, and the controller is further adapted to send an alert to the server if the first distance between the first tag and the first transceiver does not change for more than a threshold amount of time.

The patient support apparatus, in some aspects, further includes a second transceiver coupled to a second location on the patient support apparatus and a third transceiver coupled to a third location on the patient support apparatus. When so included, the controller may use RF communication between the second transceiver and the first tag to determine a second distance between the first tag and the second transceiver, and to use RF communication between the third transceiver and the first tag to determine a third distance between the first tag and the third transceiver.

In some aspects, the patient support apparatus further comprises a memory in which relative positions of the first, second, and third locations are stored, and the controller is adapted to determine a first three dimensional position of the first tag using the first, second, and third distances and the relative positions of the first, second, and third locations.

The patient support apparatus, in some aspects, further includes a control panel having a first exit detection control in communication with the controller. When the user activates the first exit detection control, the controller is adapted to monitor the first three dimensional position of the first tag and to issue an exit alert if the first three dimensional position of the first tag travels outside of a first volume of space.

The control panel, in some aspects, includes a second exit detection control in communication with the controller. When the user activates the second exit detection control, the controller is adapted to monitor the first three dimensional position of the first tag and to issue the exit alert if the first three dimensional position of the first tag travels outside of a second volume of space. The second volume of space is different from the first volume of space.

In some aspects, the first transceiver is adapted to receive an identifier from the first tag, and the controller is adapted to use the identifier to determine that the first tag is coupled to the patient.

The controller, according to some aspects, is adapted to use RF communication between the first transceiver and a second tag to determine a fourth distance between the second tag and the first transceiver, to use RF communication between the second transceiver and the second tag to determine a fifth distance between the second tag and the second transceiver, and to use RF communication between the third transceiver and the second tag to determine a sixth distance between the second tag and the third transceiver.

The controller may also be adapted to determine a second three dimensional position of the second tag using the fourth, fifth, and sixth distances and the relative positions of the first, second, and third locations.

The controller, in some aspects, is further adapted to determine if the second three dimensional position is located within a predetermined volume of space defined in a fixed relationship to the patient support apparatus.

The controller, in some aspects, is adapted to automatically associate the second tag with the patient support apparatus if the second three dimensional position is inside the predetermined volume of space, and to not automatically associate the second tag with the patient support apparatus if the second three dimensional position is outside the predetermined volume of space.

The patient support apparatus, in some aspects, further includes a fourth transceiver adapted to communicate with server, and the controller is adapted to send a message to the server indicating that the second tag is associated with the patient support apparatus if the second three dimensional position is inside the predetermined volume of space.

The first, second, and third transceivers, in some embodiments, are adapted to communicate with a wall unit affixed to a wall of a healthcare facility at a known location, and the controller is adapted to determine a position of the patient support apparatus within the healthcare facility using communications between the wall unit and the first, second, and third transceivers.

The controller, in some aspects, is adapted to receive an identifier from the second tag, and the controller is further adapted to use the identifier to determine a type of medical device to which the second tag is attached.

In some aspects, the patient support apparatus includes a control panel including a first exit detection control in communication with the controller. When the user activates the first exit detection control, the controller is adapted to monitor the first three dimensional position of the first tag and to issue an exit alert if the three dimensional position of the first tag travels outside of a first volume of space. The first volume of space being different from the predetermined volume of space.

The control panel, in some aspects, further includes a second exit detection control in communication with the controller. When the user activates the second exit detection control, the controller is adapted to monitor the three dimensional position of the first tag and to issue an exit alert if the three dimensional position of the first tag travels outside of a second volume of space. The second volume of space is different from both the first volume of space and the predetermined volume of space.

The control panel, in some aspects, includes a patient monitoring control in communication with the controller. When the user activates the patient monitoring control, the controller is adapted to monitor the first three dimensional position of the first tag and determine a cumulative amount of movement of the first tag.

In some aspects, the patient support apparatus further includes a fourth transceiver adapted to communicate with server, and wherein the controller is adapted to send a message to the server if the cumulative amount of movement of the first tag over a first period of time does not exceed a first threshold. The first period of time and first threshold may be selected to assist in preventing bed sores from developing on the patient.

Alternatively, or additionally, the controller may be adapted to send a message to the server if the cumulative amount of movement of the first tag over a second period of time exceeds a second threshold. The second period of time and the second threshold may be selected to assist in identifying patient restlessness.

In some aspects, the controller is further adapted to use the first three dimensional position of the first tag to determine if the patient has been turned by a caregiver. The controller may further be adapted to a message to a server indicating if the patient has been turned by the caregiver.

According to another aspect of the present disclosure, a patient support apparatus is provided that includes a support surface, a first transceiver, and a controller. The support surface is adapted to support a patient. The first transceiver is coupled to a first location on the patient support apparatus, and the controller is in communication with the first transceiver. The controller is adapted to use radio frequency (RF) communication between the first transceiver and a first tag to determine a first distance between the first tag and the first transceiver. The controller is further adapted to receive an identifier from the first tag and to use the identifier to determine if the first tag is coupled to a patient or to a device.

According to other aspects of the present disclosure, the patient support apparatus includes a second transceiver coupled to a second location on the patient support apparatus and a third transceiver coupled to a third location on the patient support apparatus. The controller may be adapted to use RF communication between the second transceiver and the first tag to determine a second distance between the first tag and the second transceiver, and to use RF communication between the third transceiver and the first tag to determine a third distance between the first tag and the third transceiver.

In some aspects, the patient support apparatus further includes a memory in which relative positions of the first, second, and third locations are stored, and the controller is adapted to determine a first three dimensional position of the first tag using the first, second, and third distances and the relative positions of the first, second, and third locations.

In some aspects, the patient support apparatus includes a control panel having a patient monitoring control in communication with the controller. When the user activates the patient monitoring control, the controller is adapted to determine if the first three dimensional position of the first tag is positioned inside or outside of a volume of space, and to display a first message on a display of the patient support apparatus if the first three dimensional position of the first tag is outside of the volume of space.

The first message may indicate that the controller is unable to detect when the patient exits the patient support apparatus.

In some aspects, only if the identifier indicates that the first tag is coupled to patient is the controller able to issue an exit alert if the first three dimensional position of the first tag travels outside of a first volume of space.

In some aspects, the controller is adapted to use RF communication between the first transceiver and a second tag to determine a fourth distance between the second tag and the first transceiver, to use RF communication between the second transceiver and the second tag to determine a fifth distance between the second tag and the second transceiver, and to use RF communication between the third transceiver and the second tag to determine a sixth distance between the second tag and the third transceiver.

The controller, in some aspects, is adapted to determine a second three dimensional position of the second tag using the fourth, fifth, and sixth distances and the relative positions of the first, second, and third locations.

In some aspects, the controller is adapted to receive a second identifier from the second tag and to use the second identifier to determine if the second tag is coupled to the patient or to a device.

The controller, in some aspects, is further adapted to determine if the second three dimensional position is positioned within a predetermined volume of space defined in a fixed relationship to the patient support apparatus.

The controller, in some aspects, is further adapted to automatically associate the second tag with the patient support apparatus if the second three dimensional position is inside the predetermined volume of space, and to not automatically associate the second tag with the patient support apparatus if the second three dimensional position is outside the predetermined volume of space.

The controller, in some embodiments, is adapted to use a fourth transceiver to send a message to the server indicating that the second tag is associated with the patient support apparatus if the second three dimensional position is inside the predetermined volume of space.

The patient support apparatus, in some aspects, further includes a control panel having a first exit detection control in communication with the controller, wherein when the user activates the first exit detection control, the controller is adapted to determine if either of the first or second identifiers indicate that the first or second tag is coupled to the patient. If neither of the first or second identifiers indicate that the first or second tag is coupled to the patient, the controller is configured to indicate on a display of the patient support apparatus that the controller is unable to detect when the patient exits the patient support apparatus.

If the controller determine, in some aspects, that at least one of the first and second tags is coupled to the patient, the controller is adapted to monitor the three dimensional position of the tag attached to the patient and to issue an exit alert if the three dimensional position of the tag attached to the patient travels outside of a first volume of space. The first volume of space is different from the predetermined volume of space.

A patient support apparatus according to another aspect of the present disclosure includes a support surface, a first transceiver, a second transceiver, a third transceiver, and a controller. The first transceiver is coupled to a first location on the patient support apparatus; the second transceiver is coupled to a second location on the patient support apparatus; and the third transceiver is coupled to a third location on the patient support apparatus. The controller is adapted to perform both of the following: (1) monitor movement of a patient positioned on the support surface using communications between a first tag attached to the patient and the first, second, and third transceivers; and (2) determine whether a second tag attached to a medical device should be associated with the patient support apparatus using communications between the second tag and the first, second, and third transceivers.

In some aspects, the controller is further adapted to use radio frequency (RF) communications between the first tag and the first, second, and third transceivers to determine a first three dimensional position of the first tag. The controller may be further adapted to use radio frequency (RF) communications between the second tag and the first, second, and third transceivers to determine a second three dimensional position of the second tag.

In some aspects, the patient support apparatus includes a control panel having a display and an exit detection control adapted to arm an exit detection system. The controller is further adapted to receive a first identifier from the first tag and to allow the exit detection system to be armed only if both of the following conditions are met: (1) the three dimensional position of the first tag is inside a first volume of space, and (2) the first identifier indicates that the first tag is able to be used for monitoring patient exits.

The controller, in some aspects, is further adapted to determine if the second three dimensional position is positioned within a volume of space defined in a fixed relationship to the patient support apparatus.

In some aspects, the controller may further be adapted to automatically associate the second tag with the patient support apparatus if the second three dimensional position is inside the volume of space, and to not automatically associate the second tag with the patient support apparatus if the second three dimensional position is outside the volume of space.

In some aspects, the controller is adapted to send a message to a server using a fourth transceiver indicating that the second tag is associated with patient support apparatus if the second three dimensional position is inside the volume of space.

In some aspects, the controller is adapted to determine at least one of the following from monitoring movement of the patient on the support surface: (1) whether the patient has exited from the patient support apparatus; or (2) how frequently the patient moves while positioned on the support surface.

In some aspects, the patient support apparatus further comprises a control panel having a patient monitoring control in communication with the controller. When the user activates the patient monitoring control, the controller is adapted to monitor the first three dimensional position of the first tag and determine a cumulative amount of movement of the first tag. The cumulative amount of movement may be used by the controller to determine if the patient is at risk of developing bed sores and/or if the patient is experiencing a bout of restlessness.

In some aspects, the patient support apparatus further comprises a fourth transceiver adapted to communicate with server, and the controller is adapted to send a message to the server indicating if the patient has been turned by the caregiver.

Before the various aspects of the disclosure are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus of FIG. 1;

FIG. 4 is a perspective view of the patient support apparatus and a first type of wall unit that is used for automatically detecting the location of a patient support apparatus;

FIG. 5 is a block diagram of the patient support apparatus, wall unit, a plurality of tags, and computer network of FIG. 4;

FIG. 6 is a perspective view of the patient support apparatus and a second type of wall unit that is used for automatically detecting the location of the patient support apparatus;

FIG. 7 is a block diagram of the patient support apparatus, wall unit, and computer network of FIG. 6;

FIG. 8 is a diagram of the patient support apparatus illustrating different thresholds used for determining the location of the patient support apparatus and one or more tags;

FIG. 9 is an illustrative patient monitoring screen that may be displayed on one or more displays of the patient support apparatus;

FIG. 10 is an illustrative exit detection screen that may be displayed in response to a user selecting the “exit detection” option on the screen of FIG. 9;

FIG. 11 is an illustrative bed sore monitoring screen that may be displayed in response to a user selecting the “bed sore” option on the screen of FIG. 9;

FIG. 12 is an illustrative patient turn monitoring screen that may be displayed in response to a user selecting the “turning” option on the screen of FIG. 9;

FIG. 13 is an illustrative restlessness monitoring screen that may be displayed in response to a user selecting the “restlessness” option on the screen of FIG. 9; and

FIG. 14 is an illustrative tag placement selection screen that may be displayed on one or more of the displays of the patient support apparatus.

### DETAILED DESCRIPTION OF THE EMBODIMENTS

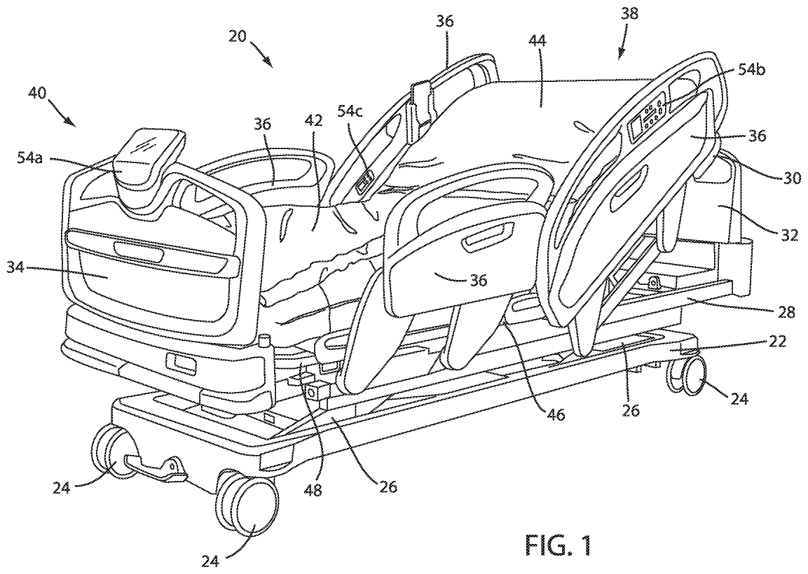

An illustrative patient support apparatus 20 according to an embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, an operating table, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. In some embodiments, the mattress 42 includes one or more inflatable bladders that are controllable via a blower, or other source of pressurized air. In at least one embodiment, the inflation of the bladders of the mattress 42 is controllable via electronics built into patient support apparatus 20. In one such embodiments, mattress 42 may take on any of the functions and/or structures of any of the mattresses disclosed in commonly assigned U.S. Pat. No. 9,468,307 issued Oct. 18, 2016, to inventors Patrick Lafleche et al., the complete disclosure of which is incorporated herein by reference. Still other types of mattresses may be used.

Support deck 30 is made of a plurality of sections, some of which are pivotable about generally horizontal pivot axes. In the embodiment shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions, such as, but not limited to, that described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may include the same, or nearly the same, structures as the Model 3002 S3 bed manufactured and sold by Stryker Corporation of Kalamazoo, Michigan. This construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. In still another embodiment, the mechanical construction of patient support apparatus 20 may include the same, or nearly the same, structure as the Model 3009 Procuity MedSurg bed manufactured and sold by Stryker Corporation of Kalamazoo, Michigan. This construction is described in greater detail in the Stryker Maintenance Manual for the 3009 Procuity MedSurg bed (publication 3009-009-002, Rev. A.0), published in 2020 by Stryker Corporation of Kalamazoo, Michigan.

It will be understood by those skilled in the art that patient support apparatus 20 can be designed with still other types of mechanical constructions, such as, but not limited to, those described in commonly assigned, U.S. Pat. No. 7,690,59 issued Apr. 6, 2010, to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The overall mechanical construction of patient support apparatus 20 may also take on still other forms different from what is disclosed in the aforementioned references provided the patient support apparatus includes the functions and features discussed in greater detail below.

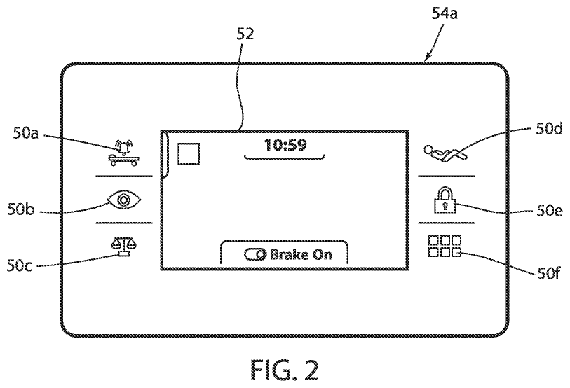

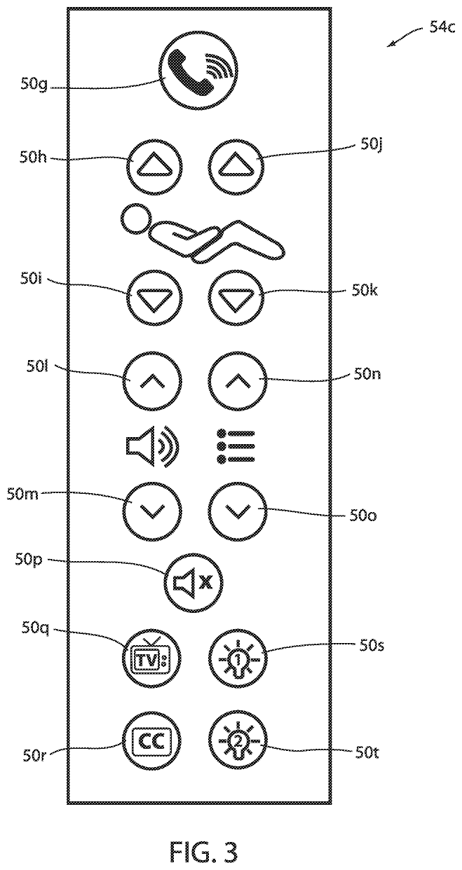

Patient support apparatus 20 further includes a plurality of control panels 54 that enable a user of patient support apparatus 20, such as a patient and/or an associated caregiver, to control one or more aspects of patient support apparatus 20. In the embodiment shown in FIG. 1, patient support apparatus 20 includes a footboard control panel 54a, a pair of outer siderail control panels 54b (only one of which is visible), and a pair of inner siderail control panels 54c (only one of which is visible). Footboard control panel 54a and outer siderail control panels 54b are intended to be used by caregivers, or other authorized personnel, while inner siderail control panels 54c are intended to be used by the patient associated with patient support apparatus 20. Each of the control panels 54 includes a plurality of controls 50 (see, e.g. FIGS. 2-3), although each control panel 54 does not necessarily include the same controls and/or functionality.

Among other functions, controls 50 of control panel 54a allow a user to control one or more of the following: change a height of support deck 30, raise or lower head section 44, activate and deactivate a brake for wheels 24, arm and disarm one or more patient monitoring functions (discussed below), change various settings on patient support apparatus 20, view the current location of the patient support apparatus 20 as determined by the location detection system discussed herein, view what device—if any—the patient support apparatus 20 has associated itself with, view the position of any tags that are positioned within the vicinity of the patient support apparatus 20 (as discussed in greater detail below), and perform other actions. One or both of the inner siderail control panels 54c also include at least one control that enables a patient to call a remotely located nurse (or other caregiver). In addition to the nurse call control, one or both of the inner siderail control panels 54c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 54c include, but are not limited to, the volume, the channel, the closed-captioning, and/or the power state of the television. With respect to the room and/or night lights, the features that may be controlled by one or more controls 50 on control panel 54c include the on/off state and/or the brightness level of these lights.

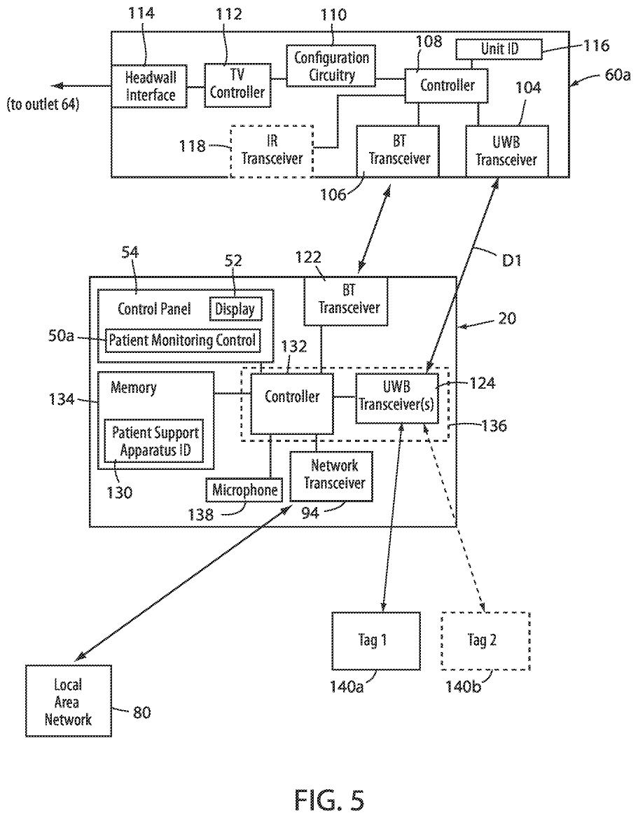

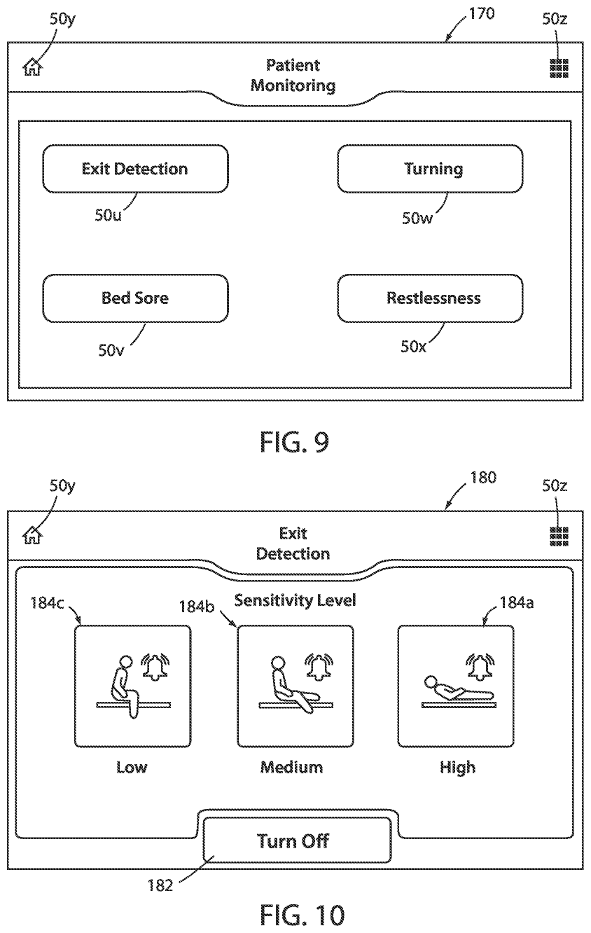

Control panel 54a includes a display 52 (FIG. 2) configured to display a plurality of different screens thereon. Surrounding display 52 are a plurality of navigation controls 54a-f that, when activated, cause the display 52 to display different screens on display 52. More specifically, when a user presses navigation control 54a, control panel 54a displays a patient monitoring screen on display 52 that includes one or more icons that, when touched, control various patient monitoring functions of patient support apparatus 20. One example of such a patient monitoring screen is shown in FIG. 9 and allows a user to arm and disarm a patient monitoring system 136 (FIGS. 5 & 7) that may monitor any one or more of the following: patient exits, bed sore possibilities, patient turns, and/or patient restlessness. These functions are described in greater detail below with respect to FIGS. 9-14.

When a user presses navigation control 54b (FIG. 2), control panel 54 displays a patient support apparatus monitoring control screen that includes a plurality of control icons that, when touched, control an onboard monitoring system that monitors one or more components, features, and/or other aspects of patient support apparatus 20. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20 for monitoring parameters of the patient support apparatus 20.

When a user presses navigation control 54c, control panel 54a displays a scale control screen that includes a plurality of control icons that, when touched, control the scale system of patient support apparatus 20. Such a scale system may include any of the same features and functions as, and/or may be constructed in any of the same manners as, the scale systems disclosed in commonly assigned U.S. patent application No. 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors that are utilized by the patient monitoring system 136, in some embodiments, or it may utilize one or more different sensors. Other scale systems besides those mentioned above in the '254 and '954 applications may alternatively be included within patient support apparatus 20.

When a user presses navigation control 50a, control panel 54 displays a motion control screen that includes a plurality of control icons that, when touched, control the movement of various components of patient support apparatus 20, such as, but not limited to, the height of litter frame 28 and the pivoting of head section 44. In some embodiments, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 50e, control panel 54a displays a motion lock control screen that includes a plurality of control icons that, when touched, control one or more motion lockout functions of patient support apparatus 20. Such a motion lockout screen may include any of the features and functions as, and/or may be constructed in any of the same manners as, the motion lockout features, functions, and constructions disclosed in commonly assigned U.S. patent application Ser. number 16/721, 133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

When a user presses on navigation control 50f, control panel 54a displays a menu screen that includes a plurality of menu icons that, when touched, bring up one or more additional screens for controlling and/or viewing one or more other aspects of patient support apparatus 20. Such other aspects include, but are not limited to, diagnostic and/or service information for patient support apparatus 20, mattress control and/or status information, configuration settings, location information, medical device association information, and other settings and/or information. One example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 50f allows a user to navigate to a screen that enables a user to configure the communication settings between patient support apparatus 20 and one or more wall units (discussed more below). Examples of the type of communication settings that may be configured in this manner are disclosed in, and illustrated in FIGS. 9-15 of, commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION, the complete disclosure of which is incorporated herein by reference.

For all of the navigation controls 54a-f (FIG. 2), screens other than the ones specifically mentioned above may be displayed on display 52 in other embodiments of patient support apparatus 20 in response to a user pressing these controls. Thus, it will be understood that the specific screens mentioned above are merely representative of the types of screens that are displayable on display 52 in response to a user pressing on one or more of navigation controls 54a-f. It will also be understood that, although navigation controls 54a-f have all been illustrated in the accompanying drawings as dedicated controls that are positioned adjacent display 52, any one or more of these controls 54a-f could alternatively be touchscreen controls that are displayed at one or more locations on display 52. Still further, although controls 54a-f have been shown herein as buttons, it will be understood that any of controls 54a-f could also, or alternatively, be switches, dials, or other types of non-button controls.

FIG. 3 illustrates one example of a patient control panel 54c that may be incorporated into patient support apparatus 20 and positioned at a location on patient support apparatus 20 that is convenient for a patient to access while supported on support deck 30, such as on an interior side of one of the siderails 36. Control panel 54c includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise Fowler section 44 upwardly. A Fowler-down control 50i, when pressed by the patient, causes the motorized actuator to lower Fowler section 44 downwardly. A gatch-up control 50j, when pressed by the patient, causes another motorized actuator to raise a knee section of support deck 30, while a gatch-down control 50k causes the motorized actuator to lower the knee section of support deck 30.

A volume-up control 50l, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 50o, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some embodiments, the first light is a reading light and the second light is a room light, both of which are positioned off-board the patient support apparatus 20.

It will be understood that not only the number of controls 50 on control panel 54c, but also the functions of the controls 50 on control panel 54c, the layout of the controls 50 on control panel 54c, and/or other aspects of control panel 54c may be modified from what is shown in FIG. 3. In some embodiments, control panel 54c is implemented on a pendant controller that includes a cable that is plugged into a port on patient support apparatus 20. In other embodiments, one or more of the controls 50 of control panel 54c may be omitted, augmented, and/or split amongst other controls panels and/or locations. Still other manners of implementing control panel 54c are also possible.

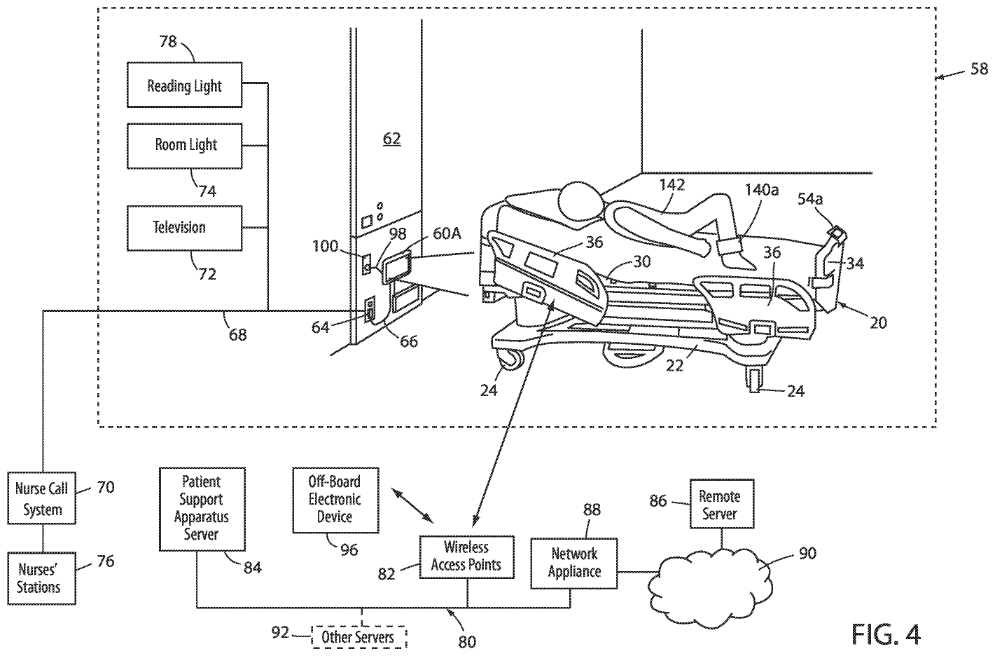

FIG. 4 illustrates a system for determining the location of patient support apparatus 20 when positioned within a room 58 of a conventional healthcare facility, such as, but not limited to, a hospital. This location detection system includes patient support apparatus 20, one or more wall units 60 (which may be of two types, 60a or 60b, as will be discussed in more detail below) and, in some aspects, a remote server, such as patient support apparatus server 84. Wall units 60 are positioned at known and fixed locations within the healthcare facility in which patient support apparatus 20 is positioned. As will be discussed in greater detail below, wall units 60 are adapted to determine how far away one or more patient support apparatuses 20 are from the wall unit 60 and/or they are adapted to allow patient support apparatuses 20 to determine how far away they are positioned from the wall unit 60.

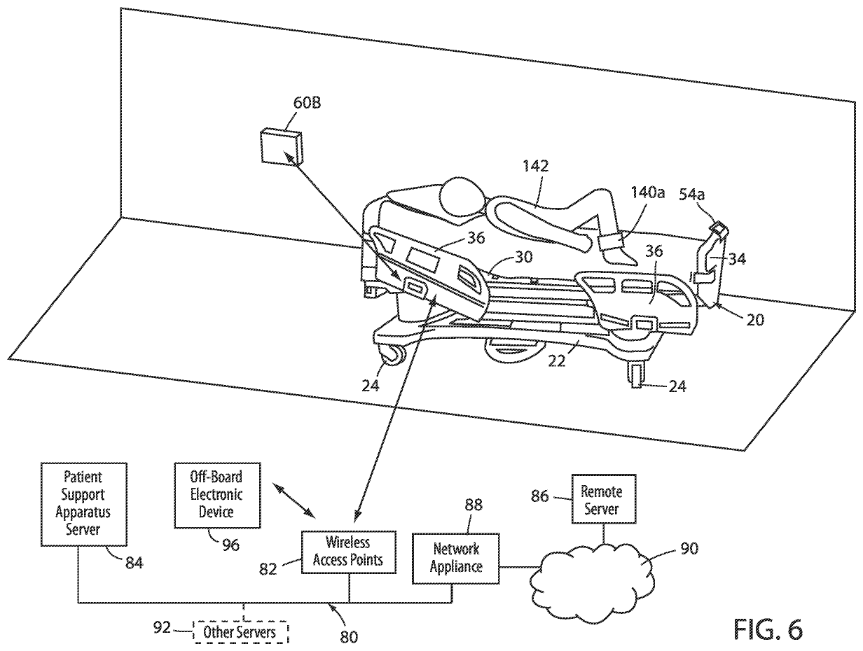

The location detection system described herein may utilize two different types of wall units 60: linked wall units 60a and unlinked wall units 60b. One example of a linked wall unit 60a is shown in FIG. 4. One example of an unlinked wall unit 60b is shown in FIG. 6. Wall units 60a and 60b differ from each other in that linked wall units 60a are adapted to communicate with a conventional communication outlet 64 that is typically built into one or more walls of a healthcare facility. That is, wall units 60a are communicatively linked to a conventional communication outlet 64. Unlinked wall units 60b are not adapted to communicate with such communication outlets 64, and are therefore not linked to a nearby communications outlet 64. Both wall units 60a and 60b are adapted to provide location information to a patient support apparatus. Linked wall units 60a, however, are also adapted to serve as a communication conduit for routing communications between patient support apparatus 20 and one or more devices and/or systems that are communicatively coupled to communication outlet 64 (e.g. room devices 72, 74, 78, and/or nurse call system 70). Unlinked wall units 60b, in contrast, are not necessarily adapted to serve as communication conduits between patient support apparatus 20 and any other electronic structures. In general, linked wall units 60a are typically positioned in patient rooms of the healthcare facility where one or more communication outlets 64 are typically present, while unlinked wall units 60b are typically positioned in locations outside of patient rooms, such as hallways, maintenance areas, and/or other areas. Unless explicitly stated otherwise, references to “wall units 60” made herein refer to both wall units 60a and 60b.

As shown in FIG. 4, linked wall units 60a are adapted to be mounted to a wall 62, such as a headwall of a patient room 58 within the healthcare facility. The headwall of a conventional healthcare facility room 58 typically includes a conventional communications outlet 64 physically integrated therein. Communications outlet 64 is adapted to receive a nurse call cable 66 that physically connects at its other end either to patient support apparatus 20 (not shown) or to wall unit 60a (shown in FIG. 4). In many healthcare facilities, communication outlet 64 includes a 37-pin connector, although other types of connectors are often found in certain healthcare facilities. As will be discussed in greater detail below, linked wall unit 60a and nurse call cable 66 allow patient support apparatus 20 to communicate with a nurse call system, and one or more room devices positioned within room 58.

Communication outlet 64 is electrically coupled to one or more cables, wires, or other conductors 68 that electrically couple the communication outlet 64 to a nurse call system 70 and one or more conventional room devices, such as a television 72, a room light 74, and/or a reading light 76. Conductors 68 are typically located behind wall 62 and not visible. In some healthcare facilities, conductors 68 may first couple to a room interface circuit board that includes one or more conductors 68 for electrically coupling the room interface circuit board to room device 72, 74, 78 and/or nurse call system 70. Still other communicative arrangements for coupling communication outlet 64 to nurse call system 70 and/or one or more room devices 72, 74, 78 are possible.

Nurse call cable 66 (FIG. 4) enables patient support apparatus 20 to communicate with nurse call system 70 and/or room devices 72, 74, 78. A patient supported on patient support apparatus 20 who activates a nurse call control (e.g. 50g; see FIG. 3) on patient support apparatus 20 causes a signal to be wirelessly sent from patient support apparatus 20 to linked wall unit 60a, which in turn conveys the signal via nurse call cable 66 to the nurse call system 70, which forwards the signal to one or more remotely located nurses (e.g. nurses at one or more nurse's stations 76). If the patient activates one or more room device controls (e.g. controls 50l-t; see FIG. 3), one or more wireless signals are conveyed to linked wall unit 60a, which in turn sends appropriate signals via nurse call cable 66 to communication outlet 64 and the room device 72, 74, 78 that change one or more features of these devices (e.g. the volume, channel, on/off state, etc.).

As is also shown in FIG. 4, patient support apparatus 20 is further configured to communicate with a local area network 80 of the healthcare facility. In the embodiment shown in FIG. 4, patient support apparatus 20 includes a wireless network transceiver 94 (FIG. 5) that communicates wirelessly with local area network 80. Network transceiver 94 is, in at least some embodiments, a WiFi transceiver (e.g. IEEE 802.11) that wirelessly communicates with one or more conventional wireless access points 82 of local area network 80. In other embodiments, network transceiver 94 may be a wireless transceiver that uses conventional 5G technology to communicate with network 80, one or more servers hosted thereon, and/or other devices. In some embodiments, network transceiver 94 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In some embodiments, network transceiver 94 is a wired transceiver that is adapted to allow patient support apparatus 20 to communicate with network 80 via a wired connection, such as an Ethernet cable that plugs into an Ethernet port (e.g. an RJ-45 style port, an 8P8C port, etc.) built into patient support apparatus 20. In still other embodiments, patient support apparatus 20 includes both a wired transceiver 94 for communicating with network 80 via a wired connection and a wireless transceiver 94 for wirelessly communicating with network 80.

Patient support apparatus 20 is configured to communicate with one or more servers on local area network 80 of the healthcare facility. One such server is a patient support apparatus server 84. Patient support apparatus server 84 is adapted, in at least one embodiment, to receive status information from patient support apparatuses 20 positioned within the healthcare facility and distribute this status information to caregivers, other servers, and/or other software applications. As will be discussed in greater detail below, server 84 may also be configured to receive data from one or more tags that are positioned within one or more volumes of space defined around patient support apparatus 20. The tags may be attached to the patient and/or one or more devices. In some embodiments where data from devices is collected, the data from one or more of tags may be forwarded to one or more other servers 92 on network 80 (and/or one or more electronic devices 96), such as a caregiver assistance server and/or a caregiver assistance software application, as will also be discussed in greater detail below.

In some embodiments, patient support apparatus server 84 is configured to communicate at least some of the patient support apparatus status data and/or the data from the tagged items that it receives from patient support apparatuses 20 to a remote server 86 that is positioned geographically remotely from the healthcare facility. Such communication may take place via a conventional network appliance 88, such as, but not limited to, a router and/or a gateway, that is coupled to the Internet 90. The remote server 86, in turn, is also coupled to the Internet 90, and patient support apparatus server 84 is provided with the URL and/or other information necessary to communicate with remote server 86 via the Internet connection between network 80 and server 86.

In some alternative embodiments, patient support apparatus 20 may be configured to communicate directly with one or more cloud-based servers, such as remote server 86, without utilizing patient support apparatus server 84. That is, in some embodiments, patient support apparatuses 20 may be configured to communicate directly with a remote server without relying upon any locally hosted servers (e.g. servers hosted on network 80). In one such embodiment, patient support apparatus 20 utilizes Microsoft's Azure could computing service to directly connect to one or more remote servers 86 without utilizing server 84. In some such embodiments, network appliance 88 may be a router configured to support such direct connections. Still other types of direct-to-cloud connections may be utilized with one or more of patient support apparatuses 20.

Patient support apparatus server 84 is also configured to determine the location of each patient support apparatus 20, or receive the location of each patient support apparatus 20 from the patient support apparatuses 20. In some embodiments, patient support apparatus server 84 determines the room number and/or bay area of each patient support apparatus 20 that is positioned within a room 58, as well as the location of patient support apparatuses 20 that are positioned outside of a room 58, such as, those that may be positioned in a hallway, a maintenance area, or some other area. In general, patient support apparatus server 84 may be configured to determine the position of any patient support apparatus 20 that is positioned within communication range of one or more wall units 60, as will be discussed in greater detail below.

It will be understood that the architecture and content of local area network 80 will vary from healthcare facility to healthcare facility, and that the example shown in FIG. 4 is merely one example of the type of network a healthcare facility may be employ. Typically, one or more additional servers 92 will be hosted on network 80 and one or more of them may be adapted to communicate with patient support apparatus server 84. For example, an electronic health record server will typically be present in any healthcare facility, and in some embodiments discussed herein, it will be in communication with patient support apparatus server 84 in order to receive patient data that is to be recorded in a patient's health record (e.g. weight readings taken from the scales built into patient support apparatuses 20; therapies provided to patients using a powered mattress 42 onboard patient support apparatuses 20, data from one or more tags and/or medical devices that are determined to be associated with the patient assigned to patient support apparatus 20, etc.). Local area network 80 will also typically allow one or more electronic devices 96 to access the local area network 80 via wireless access points 82. Such electronic devices 96 include, but are not limited to, smart phones, tablet computers, portable laptops, desktop computers, smart televisions, and other types of electronic devices that include a WiFi capability and that are provided with the proper credentials (e.g. SSID, password, etc.) to access network 80 (and, in at least some situations, patient support apparatus server 84).

Linked wall units 60a are adapted to wirelessly receive signals from patient support apparatus 20 and deliver the signals to communications outlet 64 in a manner that matches the way the signals would otherwise be delivered to communications outlet 64 if a conventional nurse call cable 66 were connected directly between patient support apparatus 20 and communications outlet 64. Linked wall units 60a are also adapted to transmit signals received from communications outlet 64 to patient support apparatus 20 via a Bluetooth transceiver 106 and/or a UWB transceiver 104 (FIG. 5). Thus, patient support apparatus 20 and linked wall unit 60a cooperate to send signals to, and receive signals from, communications outlet 64 in a manner that is transparent to communications outlet 64 such that outlet 64 cannot detect whether it is in communication with patient support apparatus 20 via a wired connection or it is in communication with patient support apparatus 20 via a wireless connection between patient support apparatus 20 and linked wall unit 60a (the latter of which is in wired communication with outlet 64). In this manner, a healthcare facility can utilize the wireless communication abilities of one or more patient support apparatuses 20 without having to make any changes to their existing communication outlets 64.

As noted, in addition to sending signals received from patient support apparatus 20 to communications outlet 64, linked wall units 60a are also adapted to forward signals received from communications outlet 64 to patient support apparatus 20. Linked wall units 60a are therefore adapted to provide bidirectional communication between patient support apparatus 20 and communications outlet 64. This bidirectional communication includes, but is not limited to, communicating command signals from any of controls 50 and/or from any of electronic devices 96 to corresponding room devices 72, 74, and/or 78 and communicating audio signals between a person supported on patient support apparatus 20 and a caregiver positioned remotely from patient support apparatus 20. The audio signals received by wall units 60 from a microphone on patient support apparatus 20 are forwarded to communications outlet 64 (for forwarding to nurse call system 70), and the audio signals of a remotely positioned nurse that are received at communications outlet 64 (from nurse call system 70) are forwarded to a speaker onboard patient support apparatus 20. Audio signals from a television 72 may also be communicated to the patient support apparatus via linked wall unit 60a.

Nurse call cable 66, in some embodiments, includes a conventional 37 pin connector on each end, one of which is adapted to be inserted into outlet 64 and the other one of which is adapted to be inserted into wall unit 60. Such 37 pin connections are one of the most common types of connectors found on existing walls of medical facilities for making connections to the nurse call system 70 and room devices 72, 74, and 78. Linked wall unit 60a and nurse call cable 66 are therefore configured to mate with one of the most common type of communication outlets 64 used in medical facilities. Such 37 pin connectors, however, are not the only type of connectors, and it will be understood that linked wall units 60a can utilize different types of connectors that are adapted to electrically couple to different types of nurse call cables 66 and/or different types of communication outlets 64. One example of such an alternative communications outlet 64 and cable 66 is disclosed in commonly assigned U.S. patent application Ser. No. 14/819,844 filed Aug. 6, 2015 by inventors Krishna Bhimavarapu et al. and entitled PATIENT SUPPORT APPARATUSES WITH WIRELESS HEADWALL COMMUNICATION, the complete disclosure of which is incorporated herein by reference. Still other types of communication outlets 64 and corresponding connectors may be utilized.

Linked wall unit 60a (FIG. 4) also includes an electrical cord 98 having a plug positioned at a far end that is adapted to be inserted into a conventional electrical outlet 100. Electrical cord 98 enables linked wall unit 60a to receive power from the mains electrical supply via outlet 100. It will be appreciated that, in some embodiments, linked wall unit 60a is battery operated and cord 98 may be omitted. In still other embodiments, linked wall unit 60a may be both battery operated and include cord 98 so that in the event of a power failure, battery power supplies power to linked wall unit 60a, and/or in the event of a battery failure, electrical power is received through outlet 100. Unlinked wall units 60b may also include a battery, electrical cord, or both.

In addition to any of the structures and functions described herein, wall units 60a (and 60b) are configured to communicate location data to patient support apparatus 20 that enables patient support apparatus 20 and/or patient support apparatus server 84 to determine the location of patient support apparatus 20 within the healthcare facility. In general, such location determination is carried out by wall units 60 sending a unique wall identifier (ID) to one or more patient support apparatuses 20 that are positioned in close proximity thereto. Patient support apparatus 20 is further adapted to determine its position relative to the wall unit 60. The combination of the patient support apparatus's relative position and the ID of the wall unit 60 is used either locally by patient support apparatus 20 to determine its position within the healthcare facility, or used remotely by server 84 to determine the position of the patient support apparatus 20 within the healthcare facility.

If determined remotely, patient support apparatus 20 may send its relative position information and/or the ID of the wall unit 60 (and its own unique patient support apparatus ID 130 (FIGS. 5 & 7)) to server 84. Server 84 includes a table of all of the locations of the wall units 60 (which may be generated via a surveying operation during the installation of wall units 60), and it uses that table to correlate the patient support apparatus IDs 130 and the wall unit IDs it receives to specific locations within the healthcare facility. Thus, if a particular patient support apparatus 20 (with a particular ID 130) sends a wall unit ID that corresponds to room 430, server 84 determines that that particular patient support apparatus 20 is currently located in room 430 (and/or in a specific position relative to that wall unit 50). Server 84 determines that the particular patient support apparatus 20 is in the same room (room 430 in this example) as the wall unit 60 because each patient support apparatus 20 is configured to associate itself with a particular wall unit 60 when it is positioned within relatively close proximity thereto. Further details of this location determination process are described below, as well as in commonly assigned U.S. Pat. No. 9,999,375 issued Jun. 19, 2018, to inventors Michael Hayes et al. and entitled LOCATION DETECTION SYSTEMS AND METHODS, the complete disclosure of which is incorporated herein by reference.

Wall units 60 may also perform additional functions. In some embodiments, wall units 60 (and/or patient support apparatuses 20) may perform any of the functions performed by the headwall units 76 (and/or patient support apparatuses) disclosed in commonly assigned U.S. patent application Ser. No. 16/215,911 filed Dec. 11, 2018, by inventors Alexander Bodurka et al. and entitled HOSPITAL HEADWALL COMMUNICATION SYSTEM, the complete disclosure of which is incorporated herein by reference. In some embodiments, wall units 60 and/or patient support apparatuses 20 may also, or alternatively, perform any of the same functions performed by the headwall interfaces 72 and/or patient support apparatuses disclosed in commonly assigned U.S. patent application Ser. No. 16/193,150 filed Nov. 16, 2018, by inventors Alexander Bodurka et al. and entitled PATIENT SUPPORT APPARATUSES WITH LOCATION/MOVEMENT DETECTION, the complete disclosure of which is also incorporated herein by reference. In still other embodiments, wall units 60 and/or patient support apparatuses 20 may also, or alternatively, perform any of the same functions performed by the headwall units 66 and patient support apparatuses disclosed in commonly assigned U.S. patent application Ser. No. 16/217,203 filed Dec. 12, 2018, by inventor Alexander Bodurka et al. and entitled SMART HOSPITAL HEADWALL SYSTEM, the complete disclosure of which is incorporated herein by reference.

In some embodiments, wall units 60 and/or patient support apparatuses 20 may be constructed to include any or all of the functionality of the wireless headwall units and/or patient support apparatuses disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION, the complete disclosure of which is incorporated herein by reference.

In some embodiments, wall units 60 and/or patient support apparatuses 20 may also be constructed to include any or all of the functionality of the headwall units and/or patient support apparatuses disclosed in commonly assigned U.S. patent application Ser. number 63/26,937 filed May 19, 2020, by inventors Alexander Bodurka et al. and entitled PATIENT SUPPORT APPARATUSES WITH HEADWALL COMMUNICATION, the complete disclosure of which is also incorporated herein by reference.

Still further, in some embodiments, wall units 60 and/or patient support apparatuses 20 may be constructed to include any of the features and/or functions of the headwall units 144a and/or patient support apparatuses disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference.

In some embodiments, patient support apparatus 20 and/or patient support apparatus server 84 may include any or all of the functionality of the patient support apparatuses and/or patient support apparatus servers described in any of the aforementioned commonly assigned U.S. patents and/or patent applications.

FIG. 5 depicts a block diagram of patient support apparatus 20 and linked wall unit 60a. Linked wall unit 60a includes an ultra-wideband transceiver 104, a Bluetooth transceiver 106, a wall unit controller 108, configuration circuitry 110, a television controller 112, a headwall interface 114, a unit ID 116, and, in some embodiments, an infrared transceiver 118. Bluetooth transceiver 106 is adapted to communicate with a Bluetooth transceiver 122 onboard patient support apparatus 20 using RF waves in accordance with the conventional Bluetooth standard (e.g. IEEE 802.14.1 and/or the standard maintained by the Bluetooth Special Interest Group (SIG) of Kirkland, Washington, USA). In some embodiments, transceivers 106 and 122 utilize Bluetooth Low Energy communications.

Ultra-wideband transceiver 104 is adapted to communicate with one or more ultra-wideband transceivers 124 positioned onboard patient support apparatus 20. Transceiver 104 is adapted to determine a distance D1 between itself and patient support apparatus 20. Alternatively, or additionally, transceiver may be adapted to allow transceiver 124 onboard patient support apparatus 20 to determine distance D1. In some embodiments, transceivers 104 and 124 use time of flight (TOF) computations to determine distance D1. In other embodiments, transceiver 104 and 124 may utilize other techniques for determining distance D1, either in addition to, or in lieu of, TOF computations. In some embodiments, transceivers 104, 124 may also determine an angle between patient support apparatus 20 and wall unit 60 using angular information derived from antenna arrays positions onboard transceivers 104, 124, or by using other techniques.

In some embodiments, transceivers 104, 124 are implemented as any of the Trimension™ ultra-wideband modules available from NXP Semiconductors of Austin, Texas. These modules include, but are not limited to, the Trimension™ UWB modules ASMOP1BO0N1, ASMOP1CO0R1, and/or the ASMOP1CO0A1, that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement transceivers 104 and 124.

Wall unit controller 108 is adapted to control the operation of transceivers 104, 106, configuration circuitry 110, TV controller 112, headwall interface 114, and, if included, IR transceiver 118 (FIG. 5). When infrared transceiver 118 is included, it may be included to provide backwards compatibility with patient support apparatuses 20 that are not equipped with a UWB transceiver 124. That is, some healthcare facilities may include one or more patient support apparatuses that are not equipped with any UWB transceivers 124, but that do include an IR transceiver that is adapted to communicate with IR transceiver 118. When linked wall unit 60a includes IR transceiver 118, it is able to communicate its unit ID 116 to such patient support apparatuses via IR transceiver 118, which is a short range transceiver that is configured to only communicate with an adjacent patient support apparatus when the patient support apparatus is nearby (e.g. without about five feet or so). Such an adjacent patient support apparatus 20 then communicates the received wall unit ID 116 along with its own unique ID 130 (FIGS. 5 & 7) to server 84 which, as noted previously, is able to correlate the wall unit ID 116 to a particular location with the healthcare facility. In this manner, server 84 is able to use linked wall units 60a determine the location of versions of patient support apparatuses 20 that don't have a UWB transceiver 104, but that do have an IR transceiver.

Headwall interface 114 is adapted to change the electrical state of one or more pins that are in electrical communication with communication outlet 64 (via cable 66). Headwall interface 114 changes these electrical states in response to instructions from controller 108. For example, if patient monitoring system 136 acts as an exit detection system and detects a patient exit, controller 132 sends an exit alert signal to linked wall unit 60a and controller 108 responds by instructing headwall interface 114 to change the electrical state of at least one pin that is used to signal an exit alert (or a generic priority alert) to the nurse call system 70 via communications outlet 64. In some embodiments, headwall interface 114 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCIING, the complete disclosure of which is incorporated herein by reference. Alternatively, or additionally, headwall interface 114 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. Linked wall unit 60a may also be configured to perform any of the functions of the headwall units 94 disclosed in the above-mentioned '778 patent application.

Configuration circuitry 110 and TV controller 112 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, linked wall unit 60a may be configured to perform any of the functions of the headwall units 144 disclosed in the aforementioned '508 patent application.

Patient support apparatus 20 includes a controller 132, a memory 134, a patient monitoring system 136, a microphone 138, Bluetooth transceiver 122, one or more UWB transceivers 124, and network transceiver 94 (FIG. 5). Each UWB transceiver 124 is positioned at a known location on patient support apparatus 20. This known location information is stored in memory 134 and/or elsewhere, and may be defined with respect to any suitable frame of reference that is fixed with respect to patient support apparatus 20. The known location information may include the spatial relationship between UWB transceivers 124 and/or any other components of patient support apparatus 20. For example, in some embodiments, the known location information includes the spatial relationship not only between UWB transceivers 124, but also the spatial relationships between UWB transceivers 124 and one or more of the following: the head end 38 of patient support apparatus 20, the foot end of patient support apparatus 20, the sides of patient support apparatus 20, the floor, and/or other components and/or landmarks of patient support apparatus 20. In some embodiments, this location information is used to determine the orientation of patient support apparatus 20 with respect to one or more walls 62, wall units 60, another patient support apparatus 20, and/or another object or structure within the healthcare facility.

Controller 132, as well as controller 108, may take on a variety of different forms. In the illustrated embodiment, each of these controllers is implemented as a conventional microcontroller. However, these controllers may be modified to use a variety of other types of circuits—either alone or in combination with one or more microcontrollers—such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controllers 108 and 132 when carrying out the functions described herein, as well as the data necessary for carrying out these functions, are stored in a corresponding memory that is accessible to that particular controller (e.g. memory 134 for controller 132, and a memory (not shown) for controller 108). In some embodiments, controller 132 may include and/or work with a microcontroller that is integrated into, or associated with, UWB transceiver(s) 124, and controller 108 may include and/or work with a microcontroller that is integrated into, or associated with, UWB transceiver 104.

Controller 132 utilizes transceiver(s) 124 to determine the relative position of patient support apparatus 20 with respect to one or more nearby wall units 60 (as well as the position of one or more tags 140 relative to patient support apparatus 20, as discussed more below). If patient support apparatus 20 is positioned within range of a wall unit 60, its UWB transceiver 124 communicates with the UWB transceiver(s) 104 positioned on that wall unit 60, and the two transceivers 124 and 104 exchange signals that enable them to determine the distance between themselves (i.e. distance D1 in FIG. 5). In some embodiments, UWB transceivers 104, 124 may also be configured to determine an angular relationship between themselves. The distance D1 (and angle information) in at least some embodiments is calculated by UWB transceiver 124 and controller 132 of patient support apparatus 20. In other embodiments, UWB transceiver 104 and controller 108 may calculate the distance D1 (and angle information) and forward the results of this calculation to patient support apparatus 20 (either via UWB transceiver 104 or BT transceiver 106). In either situation, patient support apparatus controller 132 is informed of the distance D1 (and, in some embodiments, as noted, the angle information) between transceiver 104 and 124. To the extent patient support apparatus 20 includes additional UWB transceivers 124, similar distance calculations are made between each of those UWB transceivers 124 and the wall unit's UWB transceiver 104.

Although FIG. 5 (and FIG. 6) only illustrate a single wall unit 60, it will be understood that a typical healthcare facility will include multiple wall units 60 positioned at different locations throughout the facility, including ones positioned within patient rooms and others positioned outside of patient rooms. Typically, at least one linked wall unit 60a will be positioned in each patient room of the healthcare facility, and if the patient room is intended to be occupied by more than one patient (e.g. it includes multiple bays), then additional linked wall units 60a may be included so that each patient support apparatus 20 will have a linked wall unit 60a positioned adjacent to each bay area in the room. Additional wall units 60, such as unlinked wall units 60b, may also be positioned at other locations through the healthcare facility.

In many locations throughout the healthcare facility, transceiver(s) 124 of an individual patient support apparatus 20 will be able to concurrently communicate with multiple wall units 60 because the communication range of the UWB transceivers 104 onboard the wall units 60 will overlap with each other. In such situations, controller 132 and/or controller 108 may utilize distances D1 between each UWB transceiver 104 and at least one of the UWB transceivers 124 positioned onboard patient support apparatus 20 to determine the location of the patient support apparatus. Such distances D1, to the extent they are not determined by controller 132 of patient support apparatus 20, are forwarded by the controllers 108 of the respective wall units 60 to patient support apparatus 20 (either via transceivers 104 or 106).

When controller 132 receives multiple distances D1 from multiple wall units 60, it may react in different manners, depending upon the particular embodiment of patient support apparatus 20 that is implemented. In a first embodiment, controller 132 forwards the multiple distances D1 to server 84 via network transceiver 94 and server 84 uses the multiple distances to determine a current location of that patient support apparatus 20 (via one or more conventional methods, such as, but not limited to, triangulation and/or trilateration). In a second embodiment, controller 132 uses the multiple distances to determine a current location of patient support apparatus 20 (via one or more of the same conventional methods) and then forwards the current location to server 84 via network transceiver 94. In another embodiment, controller 132 determines which wall unit 60 is closest to it and uses communications between only that wall unit 60 and its transceiver(s) 124 to determine its location (and thus doesn't rely on communications with multiple wall units 60 to determine its location).

The degree of specificity of the location determined using UWB transceivers 104 and 124 may vary depending upon how close patient support apparatus 20 is to a particular wall unit 60. For example, in some embodiments, controller 132 and/or server 84 conclude that a patient support apparatus 20 is in the same location as a particular wall unit 60 if patient support apparatus 20 is within a threshold location range of that wall unit 60. In such embodiments, the threshold location range may refer to an area having horizontal dimensions of approximately five by ten feet (and a height of approximately five or more feet above the ground) that is positioned directly in front of a linked wall unit 60a, although other dimensions may, of course, be used. For unlinked wall units 60b, the threshold location range may cover a greater area (and volume) of space. If the patient support apparatus 20 is positioned outside of the threshold location range of a particular wall unit 60, controller 132 and/or server 84 may determine an actual coordinate location of patient support apparatus 20 and/or report a more generalized location (e.g. outside of room 402; on the third floor, in corridor X, in the Emergency Department, etc.).

When controller 132 determines multiple distances D1 (whether measured between patient support apparatus 20 and multiple wall units 60 or between patient support apparatus 20 and only a single wall unit 60), controller 132 is adapted to either use those multiple distances D1 to determine its location, or it is adapted to forward those distances to an off-board entity (e.g. server 84) to use those multiple distances to determine its location. In either case, controller 132 and/or server 84 may determine the location of patient support apparatus 20 using a coordinate frame of reference in which the position of each wall unit 60 is also known (e.g. the current position of the patient support apparatus is (X,Y,Z) in a frame of reference in which a first wall unit 60 is positioned at (X₁, Y₁, Z₁), a second wall unit is positioned at (X₂, Y₂, Z₂), a third wall unit is positioned at (X₃, Y₃, Z₃), etc.). The current position of the patient support apparatus 20 may then be compared to the one or more threshold location ranges to determine if the patient support apparatus is positioned inside or outside of the threshold location ranges.

The distances D1 are determined by an exchange of communication signals between UWB transceivers 104 and 124. This exchange is initiated by an interrogation signal that may be sent by the UWB transceivers 104 of the wall unit 60, and/or it may be sent by the UWB transceivers 124 of the patient support apparatuses 20. The trigger for sending these interrogation signals (from either source) may simply be the passage of a predefined interval of time, in at least some embodiments. That is, in some embodiments, patient support apparatus 20 and/or wall units 60 may be configured to periodically send out an interrogation signal that will be responded to by any UWB transceivers 104 or 124 that are positioned with range of that signal. In those embodiments where patient support apparatuses 20 are configured to send out such an interrogation signal, the time intervals between the interrogation signals may be varied depending upon the location and/or other status of the patient support apparatus 20. For example, in some embodiments, patient support apparatuses 20 may be configured to send out the interrogation signals with longer timer intervals between them when the patient support apparatus is stationary, and to send out the interrogation signals with shorter time intervals between them when the patient support apparatus 20 is in motion. Indeed, in some embodiments, after patient support apparatus 20 has ceased moving, controller 132 may be configured to cease sending out such interrogation signals until it once again starts moving. In any of the aforementioned embodiments, motion of the patient support apparatus 20 may be detected in any suitable manner, such as by including one or more motion sensors on the patient support apparatus 20 (e.g. one or more accelerometers), and/or by monitoring the values of the repetitive distance measurements D1 and looking for changes indicative of movement.

The measured distances D1 (and/or angular information between wall units 60 and patient support apparatuses 20) that are generated from the communications between UWB transceivers 104, 124 may utilize Angle of Arrival (AoA) information, Time of Flight (TOF) information, Channel State Information, Time Difference of Arrival (TDoA) information, Two-Way Ranging (TWR) ranging information, and/or other information. In some embodiments, each transceiver 104, 124 includes an array of antennas that are used to generate distance and/or angular information with respect to the transceiver 104, 124 in which it is in communication. Still further, in some embodiments, transceivers 104, 124 include one or more of their own microcontrollers, and the location of transceivers 104, 124 may be determined by these internal microcontrollers without utilizing controller 132 and/or 108. In other embodiments, controllers 108 and/or 132 may work in conjunction with the microcontrollers of transceivers 104, 124 to determine their relative locations to each other.

Patient support apparatus 20 also includes, in at least some embodiments, a microphone 138 (FIG. 5) that is used to detect the voice of the patient when the patient wants to speak to a remotely positioned nurse. The patient's voice is converted to audio signals by microphone 138 and controller 132 is adapted to forward these audio signals to an adjacent communications outlet 64 positioned in wall 62 (FIG. 4). When a cable 66 is coupled between patient support apparatus 20 and outlet 64, controller 132 forwards these audio signals to outlet 64 via the cable. When no such cable 66 extends between patient support apparatus 20 and outlet 64, controller 132 wirelessly forwards these audio signals to headwall unit 60 (using transceiver 122, or in some embodiments, transceiver 124)) and controller 108 of headwall unit 60 forwards these audio signals to outlet 64. As was noted, outlet 64 is in electrical communication with a conventional nurse call system 70 that is adapted to route the audio signals to the correct nurse's station 76, and/or other location. In some embodiments, microphone 138 acts as both a microphone and a speaker. In other embodiments, a separate speaker may be included in order to communicate the voice signals received from the remotely positioned nurse. In some embodiments, the audio communication between patient support apparatus 20 and communications outlet 64 is carried out in any of the manners, and/or includes any of the structures, disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

After the installation of wall units 60a and 60b in a particular healthcare facility, the location of each wall unit 60 within that facility is recorded. In some embodiments, the coordinates of the locations of wall units 60 are recorded in a common frame of reference (or converted to a common frame of reference after recordation). Such coordinates may be three dimensional (i.e. include a height components), or they may be two dimensional (no height component). In other embodiments, a more generalized location of one or more wall units 60 is determined, rather than the precise coordinates of the wall units 60. In still other embodiments, the locations of one or more wall units 60 are determined both generally and more precisely. The generalized location of the wall units 60 may include an indication of the room, bay, area, hallway, portion of a hallway, wing, maintenance area, etc. that the wall unit 60 is positioned in. The specific location of the wall units 60, as noted, may include an X, Y, and Z coordinate within a common frame of reference.

In some embodiments, the location of each wall unit 60 (whether specific and/or general) is stored in a memory within that particular wall unit 60 and shared with the devices it communicates with (e.g. patient support apparatuses 20). In some embodiments, the location of each wall unit 60 is stored within memory 134 of each patient support apparatus 20. Still further, in some embodiments, the location of each wall unit 60 is stored within a memory accessible to server 84. Alternatively, or additionally, the location of each wall unit 60 may be stored in two or more of the aforementioned locations.

It will be appreciated that patient support apparatuses 20 are configured to communicate with wall units 60 regardless of the orientation of the patient support apparatus 20. That is, the UWB transceivers 104 and 124 are radio frequency transceivers that do not rely on line of sight communication, unlike the IR transceiver 118 (if present). Thus, the patient support apparatuses 20 do not have to be pointed in any particular direction with respect to the wall units in order for transceivers 104 and 124 to communicate. This differs from some prior art systems that use IR communication between the patient support apparatuses 20 and the wall units and that require the IR transceiver onboard the patient support apparatus to be aimed toward the wall unit in order for communication to be established.

It will also be understood that, although units 60 are referred to herein as “wall units” 60, such units do not necessarily have to be positioned on walls. That is, wall units 60 can be positioned on columns, ceilings, or any other fixed structures within the healthcare facility. It will therefore be understood that the term “wall” as used herein is not exclusive of ceilings, columns, or other fixed architectural structures.

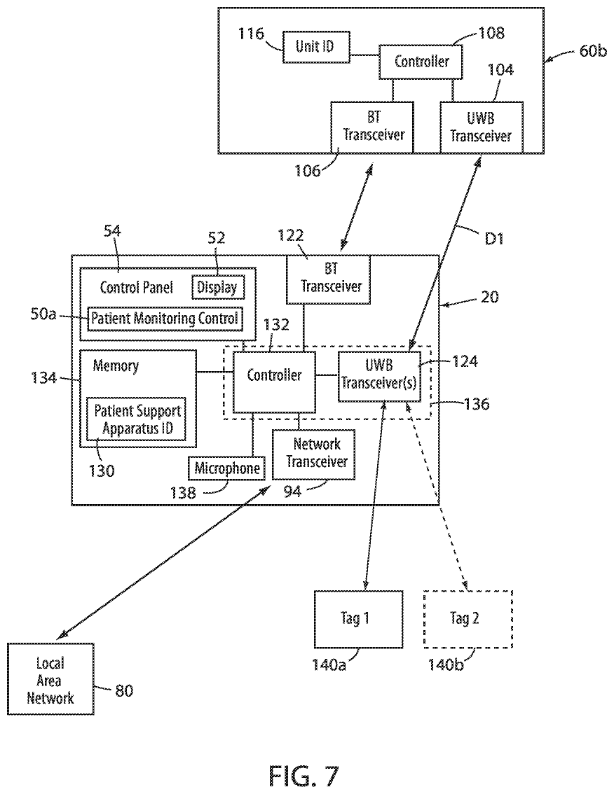

FIGS. 6 and 7 illustrate an unlinked wall unit 60b that functions to provide location information to one or more patient support apparatuses 20 in the same manner as has been described herein (and as is described further below). Unlinked wall units 60b differ from linked wall units 60a in that unlinked wall units 60b are not communicatively coupled to a communications outlet 64. As a result, unlinked wall units 60b can be positioned at any fixed location within the healthcare facility, rather than only at locations that are next to a communications outlet 64. Because such communication outlets 64 are typically only placed along the headwalls within patient rooms, linked wall units 60a are typically only positioned on the headwalls of patient rooms. Unlinked wall units 60b, however, are able to be placed in hallways, in elevators, in maintenance areas, parking garages, and/or at any other desired locations where patient support apparatuses 20 are expected to travel and where location information regarding the patient support apparatuses 20 is desired.

FIG. 6 illustrates a wall unit 60b positioned in a hallway. FIG. 7 illustrates the internal components of wall unit 60b. As can be seen in FIG. 7, wall unit 60b does not include a number of components that may be found in linked wall units 60a, such as configuration circuitry 110, television controller 112, headwall interface 114, and/or IR transceiver 118. Instead, wall unit 60b includes a controller 108, Bluetooth transceiver 106, UWB transceiver 104, and a unit ID 116. These four components work in the same manner as the corresponding components of linked wall units 60a. That is, UWB transceiver 104 is used in conjunction with transceiver 124 to determine the distance D1 between wall unit 60b and a patient support apparatus 20 (and/or to determine an angular relationship between wall unit 60b and the patient support apparatus 20). Controller 108 sends unit ID 116 to patient support apparatus 20, as well as any location and/or angular calculations that it makes in response to the communications between UWB transceiver 104 and UWB transceiver(s) 124. BT transceiver 106 may be used for any high bandwidth communications between wall unit 60b and patient support apparatus 20 that require, or are desirably carried out with, a communication channel having greater bandwidth than the communication channel between UWB transceivers 104 and 124. In some embodiments, BT transceiver 106 may be omitted entirely from unlinked wall units 60b.

Further details regarding the manner(s) in which patient support apparatus 20 may interact with one or more wall units 60 in order to determine their location, and in some cases, their orientation, are described in commonly assigned U.S. patent application Ser. No. 63/245,245 filed Sep. 17, 2021, by inventors Kirby Neihouser et al., and entitled SYSTEM FOR LOCATING PATIENT SUPPORT APPARATUSES, the complete disclosure of which is incorporated herein by reference. Wall units 60a and 60b, as well as patient support apparatuses 20, may include any of the same functionality and/or components as the wall units 60a and 60b and patient support apparatuses 20 that are described in the aforementioned '245 patent application.

In addition to being used to determine the location of patient support apparatus 20 within a healthcare facility, transceivers 124 are also used to determine the location of one or more tags 140 (FIGS. 4-8) with respect to patient support apparatus 20. For purposes of the following written description, tags 140 that are attached to a patient 142 (see FIGS. 4, 6 & 8) will be referred to as tags 140a herein, while tags 140 that are attached to a medical device will be referred to herein as tags 140b (FIGS. 5, 7, & 8). Both tags 140a and 140b may be of the same construction with their only difference being the entity to which they are attached. Alternatively, tags 140a and 140b may be constructed in different manners. For purposes of the following written description, reference to “tag 140” or “tags 140” will be understood to apply to both tags 140a and 140b.

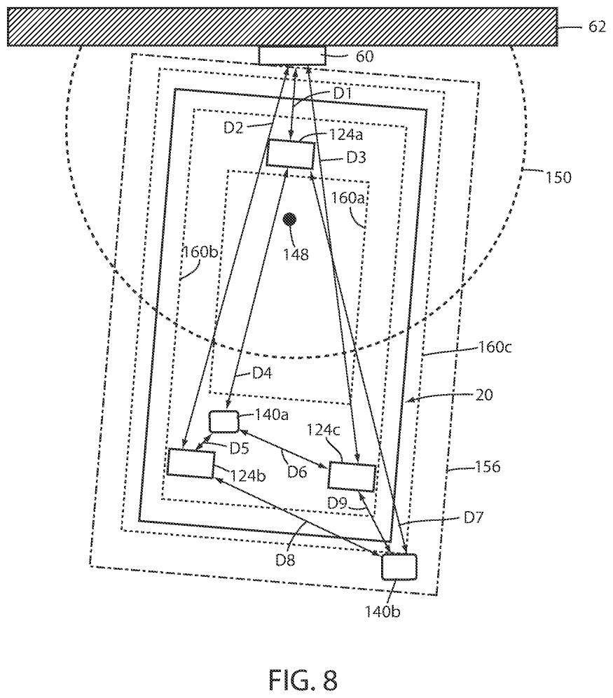

Tags 140 are adapted to use ultra-wideband (UWB) communication to communicate with UWB transceivers 124 positioned onboard patient support apparatus 20. Tags 140 may also be adapted to communicate with one or more of the UWB transceivers 104 positioned inside of wall units 60. Controller 132 uses the UWB communications between transceivers 124 and the tag(s) 140 to determine the distance between each transceiver 124 and each of the tag(s) 140. For example, as shown in FIG. 8, controller 132 uses UWB communications between transceivers 124a-c and tag 140a to determine distances D4, D5, and D6 between transceivers 124a-c, respectively, and tag 140a. Similarly, controller 132 uses UWB communications between transceivers 124a-c and tag 140b to determine distances D7, D8, and D9 between transceivers 124a-c, respectively, and tag 140b. In some embodiments, controller 132 also gathers angular information from each of the transceivers 124a-c regarding the angular relationship of each of those transceivers 124a-c to each of the tags 140. Whether such angular information is gathered or not, controller 132 uses the distance measurements to determine the three-dimensional position of each of the tags 140 that are within communication range of UWB transceivers 124 (or that are within a maximum threshold distance of these transceivers 124). In some embodiments, patient support apparatus 20 may include more than the three UWB transceivers 124 shown in FIG. 8, which may give controller 132 more precise information about the current location of each tag 140. Regardless of the number of UWB transceivers 124, controller 132 may use conventional trilateration, triangulation, and/or other algorithms for determining the three dimensional position of the tags 140.

The three dimensional position of the tags 140 is determined within a coordinate frame of reference that is local to the patient support apparatus 20, and that may or may not be correlated to the frame of reference that is used to determine the location of patient support apparatus 20 within the healthcare facility. That is, controller 132 uses a local frame of reference for determining the position of tags 140 that is based off of a fixed landmark on patient support apparatus 20, and the positions of each of the UWB transceivers 124 within that local frame of reference is known to controller 132. Because this local frame of reference is independent of the frame of reference used for determining locations within the entire healthcare facility, the three dimensional position of a tag 140 that does not move with respect to patient support apparatus 20 will retain the same three dimensional position within the local frame of reference, even if the patient support apparatus 20 is moved to different locations within the healthcare facility.

As will be discussed in greater detail below, controller 132 uses the position information from one or more patient worn-tags 140a to carry out one or more of the patient monitoring functions associated with patient monitoring control 50a (FIG. 2). Controller 132 also uses the position information from the one or more device-attached tags 140b to carry out association and/or data gathering functions regarding the devices to which the tags 140b are attached. These different uses of tags 140a and 140b are described in greater detail below.

Tags 140 may be constructed to include their own internal UWB transceiver that is similar, if not the same as, the UWB transceivers 124 and/or 104. In some embodiments, tags 140 include any of the Trimension™ ultra-wideband modules available from NXP Semiconductors of Austin, Texas. These modules include, but are not limited to, the Trimension™ UWB modules ASMOP1BOON1, ASMOP1COOR1, and/or the ASMOP1CO0A1, and that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement tags 140. Further details regarding one manner in which tags 140 may be physically constructed are provided in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference. Still other physical constructions of tags 140 may be utilized.

Patient tags 140a may be constructed to attach to a patient 142 in any suitable manner. In some embodiments, patient tags 140a are incorporated into a wristband, ankle band, or some other type of band that is wrapped around a portion of the patient's anatomy. Alternatively, patient tags 140a may include an adhesive that is used to adhere the tag 140a to the patient's skin and/or clothing. As another alternative, tags 140a may be sewn into, or otherwise integrated into, one or more of the items of clothing worn by the patient. Still other manners of securing tags 140a to the patient may be utilized.

In addition to determining the position of one or more tags 140 with respect to a local frame of reference (tied to patient support apparatus 20), controller 132 uses UWB transceivers 124 to determine the location of patient support apparatus 20 within the healthcare facility. As was described above, this location within the healthcare facility may utilize a general frame of reference that is fixed with respect to the healthcare facility, and thus independent of the local frame of reference of patient support apparatus 20. Thus, in the example shown in FIG. 8, controller 132, in addition to determining distances D4-9, also determines distances D1-D3, which are the distances between each transceiver 124 and the adjacent wall unit 60. From these distances D1-D3, as well as the wall unit ID 116 received from wall unit 60 (and, in some embodiments, additional information received from wall unit 60), controller 132 is able to determine the overall location of patient support apparatus 20 within the healthcare facility.

More particularly, controller 132 may determine the location of patient support apparatus 20 within the healthcare facility by comparing a current position of a reference point 148 onboard patient support apparatus 20 to a threshold range 150 defined with respect to an adjacent wall unit 60. The threshold range 150 defines a volume of space positioned adjacent to the wall unit 60. In some embodiments, this threshold range 150 is the same for each wall unit 60, while in other embodiments, it may vary from unit 60 to unit 60. If the reference point 148 (whose position relative to transceivers 124 is known and stored in memory 134) is positioned inside of the threshold range 150 (as shown in FIG. 8), then controller 132 determines that it should associate with the adjacent wall unit 60. If the reference point is outside of the threshold range 150 (not shown in FIG. 8), then controller 132 determines that it should not associate with the adjacent wall unit 60. Further details regarding the meaning, function, and ramifications of this association process are found in the aforementioned commonly assigned U.S. patent application Ser. No. 63/245,245, the complete disclosure of which has already been incorporated herein by reference.

FIG. 8 also illustrates a plurality of exit detection zones 160a-c. Exit detection zones 160a-c are used by controller 132 when a user has activated patient monitoring control 50a (FIG. 2) and selected the exit detection function. More specifically, when a user has activated the exit detection function, controller 132 monitors the three dimensional position of one or more of the patient tags 140a relative to one of the exit zones 160a-c (the active one of these zones 160) and issues an exit alert if the tag 140a is moved outside of that zone 160. Each of the zones 160a-c has a different size, and thus defines a different volume of space. The different zones 160a-c therefore define different levels of freedom of movement of the tag 140a.

In the example shown in FIG. 8, exit detection zone 160a defines the smallest volume of space; exit detection zone 160c defines the largest volume of space; and exit detection zone 160b defines a volume of space that is smaller than zone 160c but larger than 160a. As will be discussed in greater detail below with respect to FIG. 10, patient support apparatus 20 is configured to allow a user to select which zone 160a-c he or she wishes to use as the active zone, thereby allowing the user to change the sensitivity of the exit detection function. In other words, if the user wants to have an exit detection alert issued in response to a relatively small amount of patient movement, he or she selects zone 160a as the active zone. If the user wants to allow the patient more movement before triggering the exit alert, he or she can select zone 160b. Finally, to give the patient the most amount of freedom of movement before an exit alert is triggered, the user can select zone 160c.

It will be understood that, although zones 160a-c are illustrated in FIG. 8 as rectangles, these rectangles are actually cross sections of a volumetric shape that has a height dimension. Thus, zones 160a-c don't merely define an area in the horizontal direction, but instead define a volume of space. In general, this volume of space is defined immediately above the top surface of mattress 42 and extends outward a varying amount, depending upon the particular zone 160. In some embodiments, the position of the zone may vary as the height of litter deck 28 is raised and lowered so that, for example, the zone height may extend three feet above the top surface of mattress 42 no matter what height litter frame 28 is positioned at.

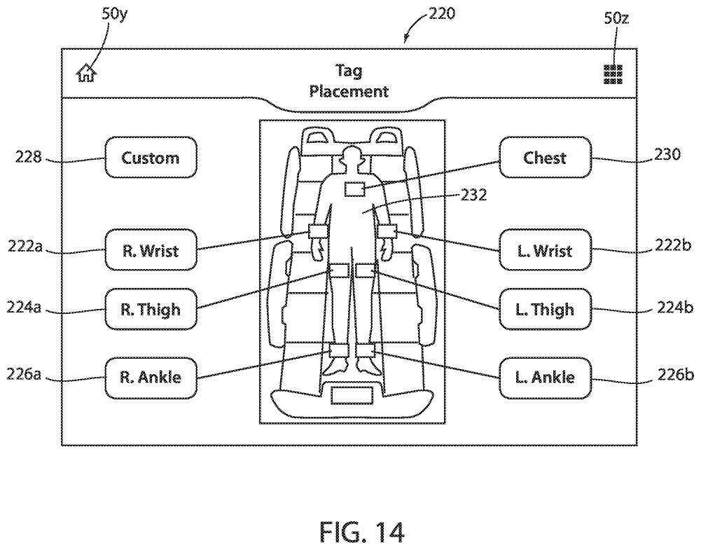

It will also be understood that, although FIG. 8 illustrates zones 160a-c as having a rectangular shape through its horizontal cross-section, this shape may be changed to a non-rectangular shape. The same is true for the shape of zones 160a-c in the Z (or height) direction-they may have a non-rectangular shape through their vertical cross-section. Additionally, although FIG. 8 illustrates all three zones 160a-c as having the same shape through their horizontal cross-sections, it will be understood that the overall shape any of zones 160a-c may vary from one or more of the other zones in its overall shape, either in addition to, or in lieu of, the variation in the volume of space defined by the different zones 160. The size and/or shape of each zone 160 may also vary depending upon what part of the patient 142's body the tag 140a is coupled to, as will be discussed in greater detail below with respect to FIG. 14. The size and/or shape of zones 160a-c may also vary depending upon how many tags 140a the patient 142 is wearing. It will also be understood that, in different embodiments, patient support apparatus 20 may include a different number of zones 160a-c than the three shown in FIG. 8, including only a single zone 160, two zones 160, or more than three zones 160.

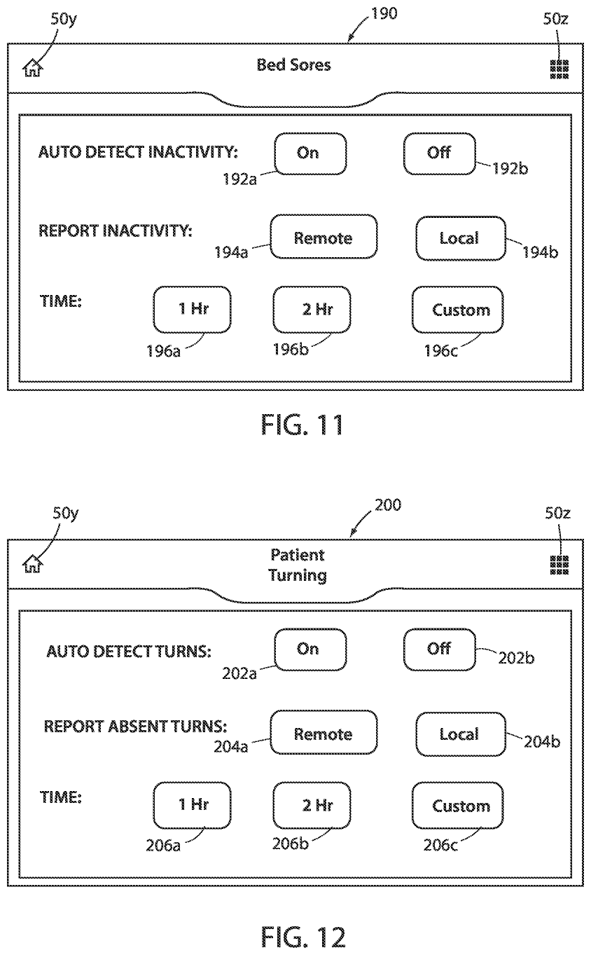

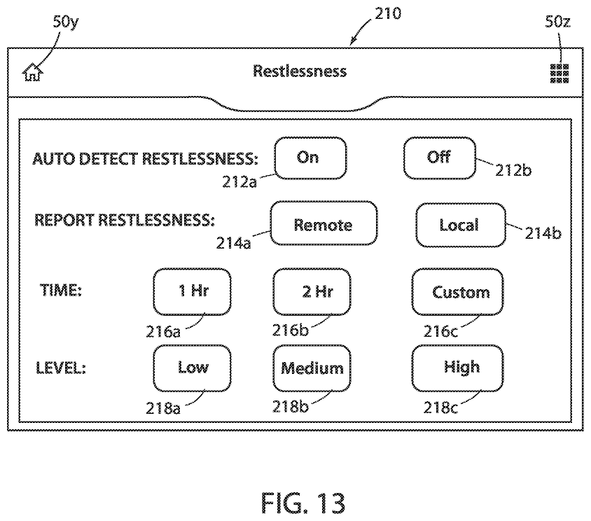

FIG. 9 illustrates a patient monitoring screen 170 that may be displayed by controller 132 on display 52 of patient support apparatus 20 in response to a user activating the patient monitoring control 54a (FIG. 2). Alternatively, or additionally, patient monitoring screen 170 may be displayed on display 52 in response to activating one or more other controls 50, and such other control(s) may be located at different positions than control 54a. Regardless of how a user navigates to patient monitoring screen 170, screen 170 allows a user to activate and/or deactivate patient monitoring system 136 (FIGS. 5 & 7). Patient monitoring system 136 is adapted to carry out a plurality of patient monitoring functions. In the example of FIG. 9, screen 170 presents the user with four patient monitoring functions: (1) an exit detection function that may be controlled via a control 50u, (2) a bed sore monitoring function that may be controlled via a control 50v, (3) a turning function that may be controlled via a control 50w, and (4) a restlessness monitoring function that may be controlled via a control 50x. When a user presses on any of controls 50u-x, controller 132 displays a control screen corresponding to the selected function on display 52. For example, in some embodiments, controller 132 is configured to display an exit detection control screen 180 (FIG. 10) in response to a user activating control 50u; to display a bed sore control screen 190 (FIG. 11) in response to a user activating control 50v; to display a turning control screen 200 (FIG. 12) in response to a user activating control 50w; and to display a restlessness control screen 210 (FIG. 13) in response to a user activating control 50x.

Patient monitoring screen 170 (FIG. 9) also includes a home control 50y and a menu control 50z. When a user activates home control 50y, controller 132 stops displaying patient monitoring screen 170 and switches to displaying a home screen (not shown). The contents of the home screen may vary from embodiment to embodiment. In some embodiments, the home screen includes one or more controls for controlling movement of the lifts 26, head section 44, and/or other components of patient support apparatus 20. When a user activates menu control 50z, controller stops displaying patient monitoring screen 170 and switches to displaying a menu screen (not shown), which may be the same as the menu screen displayed when a user presses on menu control 50f (FIG. 2). The contents of the menu screen may vary from embodiment to embodiment. In some embodiments, the menu screen includes a plurality of control options that are selectable by the user, such as, but not limited to, a settings option, a service option, a help option, a night light option, a lockout option, a history option, etc.

Exit detection control screen 180 (FIG. 10) allows a user to control the onboard patient monitoring system 136 so that it functions as an exit detection system. Exit detection control screen 180 includes an on/off control 182 and three zone selection controls 184a-c. On/off control 182 toggles between arming and disarming the exit detection function as the user presses on it. Zone selection controls 184a-c allow the user to select which zone 140a-c (FIG. 8) is going to be the active zone. In other words, zone selection controls 184a-c allow the user to select different sensitivities for the exit detection function. Zone selection control 184a corresponds to the highest sensitivity zone, which in turn corresponds to zone 160a (FIG. 8), which has the smallest volume of the three zones 160a-c. Zone selection control 184b corresponds to a medium sensitivity zone, which in turn corresponds to zone 160b (FIG. 8), which has a volume between that of zone 160a and 160c. Zone selection control 184c corresponds to the least sensitive zone, which in turn corresponds to zone 160c (FIG. 8), which has the largest volume of the zones 160a-c.

Once a user has selected a particular zone sensitivity and armed the exit detection function, controller 132 uses the repetitive three-dimensional position readings of tag 140a to monitor the patient's movement/position on support deck 30. As was described previously, if the 3-D position of tag 140a moves outside of whichever zone 160a-c the user selected via controls 184a-c (i.e. the active zone), controller 132 issues an exit alert. The exit alert may be issued through the illumination of one or more lights on patient support apparatus 20, the emission of one or more sounds from patient support apparatus 20, and/or the transmission of one or more messages off-board patient support apparatus 20 to one or more remote devices. In some embodiments, controller 132 is configured to send an exit detection message both to the wall unit 60a that it is currently associated with (if any) and to patient support apparatus server 84. The exit detection message to the wall unit 60a is sent via transceivers 122 and/or 124, while the exit detection message to server 84 is sent via transceiver 94. Server 94 may be configured to forward the exit detection message to one or more electronic devices 96 that are associated with caregivers who are assigned to caring for the particular patient assigned to that particular patient support apparatus 20.

In some embodiments, controller 132 checks one or more criteria before allowing the user to successfully arm the exit detection function of patient monitoring system 136. One criteria is the current position of the tag 140a. In order for the exit detection function to be properly armed, controller 132 checks the current three-dimensional position of the tag(s) 140a and determines whether it/they are currently positioned inside of the active zone 160. If not, controller 132 notifies the user that the tag 140a is outside of the active zone 160 and that the exit detection function cannot be armed until the tag 140a is positioned inside of the active zone 160. In some embodiments, controller 132 also checks to see if the tag 140a is the type of tag 140a that correspond to a patient worn-tag 140. In other words, controller 132 checks to see that tag 140 is not a device-attached tag, such as tags 140b. This checking step may be accomplished in different manners.

In some embodiments, each tag 140 has an ID that is used to determine whether it is attached to a person (e.g. a tag 140a) or attached to a device (e.g. a tag 140b). After transceivers 124 establish communication with tag 140, tag 140 shares this ID with the transceivers 124, which forward the ID to controller 132. Memory 134 contains data readable by controller 132 that allows controller 132 to determine from the ID whether the tag 140 is a patient tag 140a or a device tag 140b. If the tag 140 is a patient tag 140a, controller 132 allows the arming of the exit detection function to continue. If the tag 140 is a device tag 140b, controller 132 notifies the user that the tag 140b is attached to a device, and not the patient 142, and that the exit detection function cannot use that tag 140b for exit detection monitoring purposes.

In some situations, both a patient and a device tag 140a and 140b may be positioned within communication range of transceivers 124 when the user attempts to arm the exit detection function. In such situations, controller 132 may be configured to allow the arming of the exit detection function because there is at least one patient tag 140a within detection range (provided the tag 140a is currently positioned inside of the zone 160 selected by the user to be the active zone). In other situations, controller 132 may detect multiple patient tags 140a positioned inside of the active zone 160 when the exit detection function is armed. In such situations, controller 132 may be configured with logic for using multiple tags 140a for carrying out the exit detection function. That is, in some embodiments, controller 132 is configured to monitor multiple patient tags 140a and, depending on both of their 3-D positions, issue an exit alert if both off their 3-D positions satisfy certain criteria.

For example, in some embodiments, controller 132 monitors multiple patient tags 140a and only issues an exit alert if both of the tags 140a simultaneously move outside of the active zone. In other embodiments, controller 132 may be configured to monitor multiple tags 140a and to issue an exit alert only if the two tags 140a meet one or more other conditions. The other conditions may refer to a first tag 140a being outside of the active zone 160 while the other tag 140a is positioned inside (or outside) of a particular sub-zone, or they may refer to each tag 140a having its own zone 160 assigned to it, or they may refer to still other conditions, including conditions relating to the amount of time that a particular tag 140a is positioned in a particular volume of space and/or the relative position and/or orientation of the tags 140a to each other (and/or to the patient support apparatus 20).

In some embodiments, controller 132 may implement the exit detection function by utilizing additional sensor inputs beyond the current position of one or more tags 140a. For example, in some embodiments, controller 132 may implement the exit detection function by combining the 3-D position data of tags 140a with the sensor data from one or more auxiliary exit detection sensors built into patient support apparatus 20. The auxiliary exit detection sensors may include load cells that support the litter frame 28, and that determine patient movement based on changes in the readings of those load cells. An example of such auxiliary exit detection sensors, including the manner in which the outputs from such sensors may be utilized, is disclosed in commonly assigned U.S. Pat. No. 5,276,432 issued to Travis on Jan. 4, 1994, and entitled PATIENT EXIT DETECTION MECHANISM FOR HOSPITAL BED, the complete disclosure of which is incorporated herein by reference. Another example of the manner(s) in which the outputs from such auxiliary exit detection sensors may be used and incorporated into patient support apparatus 20 is disclosed in commonly assigned U.S. patent application No. 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. Still other types of auxiliary exit detection sensors may be included within patient support apparatus 20 and utilized in conjunction with tags 140a.

When using additional sensor inputs, such as those from a plurality of load cells, controller 132 may be configured to issue an exit alert only when tag 140a moves outside of the active zone 160 and the sensor readings from the auxiliary sensors, such as load cells, satisfy one or more additional criteria. In some embodiments, controller 132 calculates the center of gravity of the patient using the load cell readings and only issues the exit alert if the patient's center of gravity meets a particular criterion at the same time that the tag 140a has moved outside of the active zone. In other embodiments, the exit detection function may be carried out by controller 132 utilizing only the tags 140a and without regarding to any inputs from any load cells that may be present on the patient support apparatus 20.

Bed sore control screen 190 (FIG. 11) allows a user to control the onboard patient monitoring system 136 so that it functions as a bed sore monitoring and/or prevention system. When functioning in this manner, controller 132 apprises a caregiver of when the patient has moved an insufficient amount over a given period of time, which may lead to the development and/or exacerbation of one or more bed sores. In other words, the bed sore monitoring function informs the caregiver whether the patient is moving enough to mitigate the risk of bed sore development and/or bed sore exacerbation. Controller 132 carries out this function by monitoring the movement of the one or more patient tags 140a. In some embodiments, controller 132 detects and records the cumulative amount of movement of tag(s) 140a over a given time period and, if the cumulative amount of movement does not exceeds a particular threshold, it issues a bed sore warning notification. The notification may be sent to patient support apparatus server 84 via transceiver 94, and server 84 may then forward the notification to one or more electronic devices 96 associated with caregiver(s) assigned to the particular patient on patient support apparatus 20.

Bed sore control screen 190 (FIG. 11) includes an on control 190a, an off control 190b, a remote control 194a, a local control 194b, a 1-hour control 196a, a 2-hour control 196b, and a custom control 196c. Pressing on control 190a causes controller 132 to activate the bed sore monitoring function. Pressing off control 190b causes controller 132 to deactivate the bed sore monitoring function. Pressing remote control 194a causes controller 132 to send an alert to a remote location when an insufficient amount of movement is detected over a threshold amount of time. This remote notification includes a message sent via transceiver 94 to server 84, which as noted above, may then forward one or more notifications to one or more electronic devices 96. Pressing local control 194b causes controller 132 to issue only a local alert when an insufficient amount of movement occurs over the threshold amount of time. The local alert involves changing the status of one or more components onboard patient support apparatus 20 (e.g. changing the illumination of one or more lights and/or emitting one or more sounds).

Pressing on any of controls 196a-c allows the user to select the time period during which the threshold amount of movement must occur in order to avoid the issuance of a bed sore notification. For example, if the user presses on 1-hour control 196a, controller 132 monitors the cumulative amount of 3-D movement of tag 140a and, if that cumulative amount of movement over any given one hour time period is less than the threshold amount of movement, it issues a bed sore notification. Similarly, if the user presses on the 2-hour control 196b, controller 132 monitors the cumulative amount of 3-D movement of tag 140a and, if that cumulative amount of movement over any given two hour time period is less than the threshold amount of movement, it issues a bed sore notification. If the user presses on custom option 196c, the user is able to select any desired amount of time for controller 132 to use when carrying out this bed sore monitoring function.

Although not included on bed sore control screen 190 (FIG. 11), screen 190 may be modified to include one or more additional controls. For example, in some embodiments, control screen 190 includes one or more controls for allowing the user to select the threshold amount of movement that must occur in order to avoid a bed sore notification alert from being issued. In some embodiments, screen 190 may implement this threshold amount of movement selection function by including a low control, a medium control, and a high control. If the user selects the low control, the patient tag 140a only needs to move a relatively low amount to avoid triggering a bed sore notification. If the user selects the medium control, the patient tag 140a needs to move a greater amount in order to avoid triggering the bed sore notification. And if the user selects the high control, the patient tag 140a needs to move an even greater amount in order to avoid triggering the bed sore notification. Other manners for allowing the user to select the threshold amount of movement may also be implemented.

Screen 190 (FIG. 11), in some embodiments, also includes one or more controls for specifying further information about the locations on the patient's body for which bed sore monitoring is desired. For example, screen 190 may include options for specifying the patient's sacrum, the backs of the patient's heels, the back of the patient's head, and/or other locations. Whichever option(s) is selected, controller 132 specifically monitors movement of that location on the patient when carrying out the bed sore monitoring function. In some embodiments, a tag 140a is placed on, or near, the individual locations for which such monitoring is desired. In other embodiments, controller 132 may be configured to infer the amount of movement of a particular location on the patient's body based on the movement of another portion of the patient's body to which the tag 140a is positioned. For example, a tag 140a that is attached to the patient's ankle can be used to infer movement, or lack of movement, of the back of the patient's heel. Similarly, a tag 140a attached to the patient's torso may be used to infer movement, or lack of movement, of the patient's sacrum.

In those embodiments where monitoring system 136 allows the user to specify individual locations on the patient's body to be monitored for potential bed sore development, controller 132 may utilize a separate threshold amount of movement and/or a separate time period over which the movement, or lack of movement, is assessed for that particular location. In other words, for example, controller 132 may utilize a first threshold amount of cumulative movement of the patient's sacrum that must occur for a first amount of time in order to avoid a bed sore notification, and use a second threshold amount of cumulative movement of the patient's heels that must occur for a second amount of time in order to avoid a bed sore notification, wherein the first threshold amount of cumulative movement may be different from the second threshold amount of cumulative movement and/or the first amount of time may be different from the second amount of time. In some embodiments, controller 132 may be configured to automatically monitor different areas of the patient's anatomy for movement using different time periods and/or different thresholds without the user having to specify which of these areas are desirably monitored.

Turning control screen 200 (FIG. 12) allows a user to control the onboard patient monitoring system 136 so that it functions as a patient turning monitoring system. When functioning in this manner, controller 132 apprises a caregiver of when the patient has not been turned (either with or without caregiver assistance) over a given period of time, and/or apprises a caregiver of when a patient has been turned (either with or without caregiver assistance). In many healthcare facilities, patient care protocols require certain patients to be turned with a certain frequency, and system 136, when carrying out the patient turning function, helps the caregiver to ensure that such turning protocols are followed. As with the other functions carried out by system 136, controller 132 carries out this patient turning function by monitoring the movement and position of the one or more patient tags 140a. Depending upon the number of tags 140a that are positioned on patient 192, as well as their location, controller 132 may carry out this turning monitoring function in different manners.

For example, if a tag 140a is placed in the center of the patient's chest, controller 132 monitors the position of that tag 140a to see if the height of the tag 140a above the support deck 30 changes by a threshold amount. The threshold amount corresponds to the general change in the height of a patient's chest that is expected when a person moves from a supine position to a position lying on his or her side, or vice versa. Alternatively, or additionally, tags 140a and/or transceivers 124 may be configured to allow the orientation of tag 140a to be determined, and the orientation of tag 140a may be monitored to determine when the patient turns. For example, if the patient is in the supine position with a tag 140a on his/her chest that is facing upwards, when the patient is turned to his or her right or left side, the tag 140a will move such that it is no longer pointing upward, but is instead pointing generally horizontally to the right or the left. Still other methods for determining when the patient turns may be utilized, including the monitoring of multiple tags 140a positioned on the patient, and/or the use of load cells integrated into the patient support apparatus 20 that detect shifts in the patient's weight and/or center of gravity.

Turning control screen 200 (FIG. 12) includes an on control 202a, an off control 202b, a remote control 204a, a local control 204b, a 1-hour control 206a, a 2-hour control 206b, and a custom control 206c. Pressing on control 202a causes controller 132 to activate the turning monitoring function. Pressing off control 202b causes controller 132 to deactivate the turning monitoring function. Pressing remote control 204a causes controller 132 to send an alert to a remote location when no turn was detected over a threshold amount of time. This remote notification includes a message sent via transceiver 94 to server 84, which may then forward one or more notifications to one or more electronic devices 96. Pressing local control 204b causes controller 132 to issue only a local alert when no turn is detected over the threshold amount of time. The local alert involves changing the status of one or more components onboard patient support apparatus 20 (e.g. changing the illumination of one or more lights and/or emitting one or more sounds).

Pressing on any of controls 206a-c (FIG. 12) allows the user to select the time period during which a turn must be detected in order to avoid the issuance of a missing turn notification. For example, if the user presses on 1-hour control 206a, controller 132 monitors the 3-D movement of tag(s) 140a and, if no turn is detected within any given one hour time period, it issues a missing turn notification. Similarly, if the user presses on the 2-hour control 206b, controller 132 monitors the 3-D movement of tag(s) 140a and, if no turn is detected within any given two hour time period, it issues a missing turn notification. If the user presses on custom option 206c, the user is able to select any desired amount of time for controller 132 to use when automatically determining whether a patient turn has been missed.

Although not included on patient turning control screen 200 (FIG. 12), screen 200 may be modified to include one or more additional controls. For example, in some embodiments, control screen 200 includes one or more controls for allowing the user to select the type of turns (leftward, rightward) and/or the number of turns that must occur within the threshold period of time to avoid the issuance of a missing turn notification. Additionally, or alternatively, control screen 200 may include an option for allowing the caregiver to receive a notification every time a patient turn is detected, rather than, or in addition to, notifications of only missing turns. When selecting this option, whenever controller 132 determines that the patient has turned, it sends a turn message to patient support apparatus server 84 via transceiver 94, and server 84 reports this turn message to one or more of the electronic devices 96 that are associated with the caregiver(s) assigned to that particular patient. In this manner, caregivers are apprised of patient turns regardless of the caregiver's current location within the healthcare facility.

Restlessness control screen 210 (FIG. 13) allows a user to control the onboard patient monitoring system 136 so that it functions as a patient restlessness detection system. When functioning in this manner, controller 132 apprises a caregiver of when the patient appears to be restless for a given period of time. The monitoring of the patient's restlessness may be particularly informative of the patient's quality of sleep, pain level, and/or other factors. As with the other functions carried out by system 136, controller 132 carries out this restlessness detection function by monitoring the movement and position of the one or more patient tags 140a.

Restlessness detection control screen 210 (FIG. 13) includes an on control 212a, an off control 212b, a remote control 214a, a local control 214b, a 1-hour control 216a, a 2-hour control 216b, a custom control 216c, a low control 218a, a medium control 218b, and a high control 218c. Pressing on control 212a causes controller 132 to activate the restlessness monitoring function. Pressing off control 212b causes controller 132 to deactivate the restlessness monitoring function. Pressing remote control 214a causes controller 132 to send an alert to a remote location when restlessness is detected for a threshold amount of time. This remote notification includes a message sent via transceiver 94 to server 84, which may then forward one or more notifications to one or more electronic devices 96. Pressing local control 214b causes controller 132 to issue only a local alert when restlessness is detected for the threshold amount of time. The local alert involves changing the status of one or more components onboard patient support apparatus 20 (e.g. changing the illumination of one or more lights and/or emitting one or more sounds).

Pressing on any of controls 216a-c (FIG. 13) allows the user to select the time period during which restlessness must be detected in order to trigger the issuance of a restlessness notification. For example, if the user presses on 1-hour control 216a, controller 132 monitors the 3-D movement of tag(s) 140a and, if the cumulative amount of movement of the tag 140a over any given one hour time period exceeds a threshold, it issues a restlessness notification. Similarly, if the user presses on the 2-hour control 216b, controller 132 monitors the 3-D movement of tag(s) 140a and, if the cumulative amount of movement of the tag 140a over any given two hour time period exceeds a threshold, it issues a restlessness notification. If the user presses on custom option 216c, the user is able to select any desired amount of time for controller 132 to use when determining whether to issue a restlessness notification.

Controls 218a-c (FIG. 13) allow the user to specify different levels of cumulative movement that must occur in order for a restlessness alert to be issued. If the user presses the low option 218a, controller 132 issues the restlessness alert if a relatively small amount of cumulative movement of tag 140a occurs during the given time period. If the user presses the medium option 218b, controller 132 issues the restlessness alert if a larger amount of cumulative movement of tag 140a occurs during the given time period. And if the user presses the high option 218c, controller 132 issues the restlessness alert only if a relatively large amount of cumulative movement of tag 140a occurs during the given time period.

In some embodiments of patient support apparatus 20, controllers 132 is configured to display a tag location screen, such as tag location screen 220 (FIG. 14). Tag location screen 220 allows a user to inform controller 132 of the location of the one or more patient tags 140a on the patient's body. Controller 132 is adapted to modify the algorithms it uses for carrying out the different functions of monitoring system 136 (e.g. exit detection, bed sore monitoring, turning detection, and restlessness detection), depending upon the placement of the patient tag(s) 140a on the patient's body. Thus, for example, controller 132 is configured in some embodiments to use different sized and/or shaped exit detection zones 160 when a tag 140a is attached to the patient's wrist, as opposed to the patient's torso. Similarly, when monitoring the cumulative amount of movement of a tag 140a over a given time period (for restlessness and/or bed sore monitoring), different thresholds may be used depending upon where the tag 140a is attached to the patient's body.

Tag selection screen includes a right wrist option 222a, a left wrist option 222b, a right thigh option 224a, a left thigh option 224b, a right ankle option 226a, a left ankle option 226b, a custom option 228, a chest option 230, and a patient diagram 232. The user selects whichever of options 222-226, 230 correspond to the location of the one or more tags 140a that are attached to the patient. For example, if the patient has a first tag 140a attached to her right wrist and a second tag attached to her chest, the user presses on the right wrist option 222a and the chest option 230. This selection informs controller 132 of the location of the tag(s) 140a on the patient.

If any tag 140a is positioned on the patient in a location other than the ones corresponding to options 222, 224, 226, or 230, the user can select the custom option 228. After selecting the custom option, the user can inform controller 132 of the particular location of the tag 140a on the patient's body. In some embodiments, the user can inform controller 132 of the particular location of the tag by moving a cursor, arrow, pointer, or other structure to a desired location on patient diagram 232, and then selecting that particular location. In other embodiments, controller 132 displays a list of additional patient locations in response to the user pressing custom option 228, and user selects from this list. Still other manners of informing controller 132 of customized locations of the tag(s) 140a are possible.

In some embodiments, controller 132 may be informed of the location of tags 140a in other manners besides tag selection screen 220. In such embodiments, screen 220 may be omitted, or it may be retained as a supplemental method for informing controller 132 of the location of the patient tags 140a. One alternative method for informing controller 132 of the location of tag(s) 140a on the patient's body may utilize the IDs of each tag 140a. For example, in some embodiments, tags 140a that are attached to the patient's right wrist may have a different ID than tags that are positioned elsewhere on the patient's body. Thus, each tag 140a has an ID that identifies the location on the patient's body to which it is attached. Controller 132 may therefore determine the location of the tag(s) 140 on the patient's body through the use of the IDs that are transmitted from the tag to controller 132.

As another alternative, tag(s) 140a may be placed on all patients at a standard location, and controller 132 may be apprised of this location during the manufacture of patient support apparatus 20 (and/or during a post-manufacturing configuration step of patient support apparatus 20). In such embodiments, controller 132 knows the location of the tag(s) 140a because they are uniformly positioned at the same location on a patient as part of a healthcare facility policy.

As yet another alternative, controller 132 may be apprised of the location of the tag(s) on the patient's body by instructing the patient to move to a particular predefined position/posture, after which controller 132 determines the position of each tag 140a and automatically determines where they are positioned on the patient's body. For example, the patient may be instructed to lie flat on her back, with her hands at her side while keeping her legs flat on mattress 42. Once in this position, the user may press a button, or other control, on patient support apparatus 20, which indicates to controller 132 that the patient is currently in this position. Controller 132 then takes a snapshot of the position of each tag 140a and automatically correlates each tag 140a to a particular location of the patient's body based on the fact that the position/posture of the patient's body was known at the time of the snapshot. Thereafter, the patient is free to move to any position and controller 132 is able to discern where each tag 140a is located on that particular patient's body. Still other manners of informing controller 132 of the location of the tags 140a on the patient's body may be utilized.

It will be understood that the various monitoring functions of patient monitoring system 136 described herein can be carried out individually and/or simultaneously. Thus, if a user wishes to simultaneously use patient monitoring system 136 to monitor for the patient's exit, for bed sore prevention, for turning, and for restlessness detection, he or she can do so by selecting all four options 50u-x on screen 170 (FIG. 9). Similarly, if the user wishes to utilize only three of these functions simultaneously, or two of them, or only a single one of them, he or she can do so by selecting the appropriate control 50u-x. Of course, the user can also turn off all of these functions at any time.

It will also be understood that patient support apparatus 20 can be modified to include not only a different number of monitoring functions than the four described herein (including both fewer and lesser numbers), but also to include monitoring functions that are of a different type than those described herein. For example, in some embodiments, patient support apparatus 20 includes only the exit detection function and no bed sore monitoring, turning, or restlessness functions. In other embodiments, patient support apparatus 20 may include only another single one of these four functions. In still other embodiments, patient support apparatus 20 only includes two or three of these functions. Still further, in some embodiments, patient support apparatus 20 includes one or more types of monitoring functions that are different than the four shown in FIG. 9.

One such different monitoring function is a monitoring function that relates to the movement, or lack of movement, of a particular portion of the patient's anatomy. For example, if a patient has an injury in a particular location and it is desirable for the patient to avoid lying, or otherwise putting pressure, on that location, an appropriately positioned tag 140a can be attached to the patient and system 136 can be modified to include a monitoring function that monitors the position of that tag 140a and issues a notification if the patient moves to an undesired orientation that puts pressure on the injury. Monitoring of individual locations on the patient's body that are desirably kept immobile for certain time periods may also be performed by monitoring system 136.

Monitoring system 136 may also be configured, in some embodiments, to monitor the location of one or more tags 140b that are attached to devices, rather than to patient 142. In some embodiments, controller 132 is configured to automatically determine if a tag 140b that is coupled to a device is positioned within an association threshold 156 of patient support apparatus 20 (FIG. 8). If the tag 140b is positioned within the association threshold 156, controller 132 automatically concludes that the tag 140b is attached to a device that is to be associated with that particular patient support apparatus 20, as well as the patient assigned to that particular patient support apparatus 20. Thus, for example, if a tag 140b attached to a ventilator is positioned within association threshold 156, controller 132 concludes that the ventilator is being used with the patient onboard patient support apparatus 20. Controller 132 may then automatically take one or more additional actions in response to this determination. These additional actions include, but are not limited to, informing server 84 of the determination that the tag 140b (and its associated device) should be associated with patient support apparatus 20 (and the patient assigned thereto), forwarding data from tag 140b and/or the attached device to server 84, and/or other actions.

Association threshold 156, like exit detection zones 160a-c, defines a three-dimensional volume of space (FIG. 8). Generally speaking, association threshold 156 encompasses the volume occupied by patient support apparatus 20, as well as a certain amount of space around the perimeter of patient support apparatus 20. This perimeter space allows for the automatic association of one or devices that are customarily positioned adjacent to patient support apparatus 20, such as, but not limited to, a portable IV pole. In some embodiments, controller 132 forwards the location of tag 140b to server 84 and server 84 determines whether the tag 140b (and associated device) should be associated with that particular patient support apparatus 20.

When patient support apparatus 20 and/or server 84 associate a tagged device with a particular patient support apparatus 20, controller 132 and/or server 84 are configured to inform medical personnel (via electronic devices 96) that the tagged device is associated with a particular patient support apparatus 20 and/or with a particular patient assigned to that patient support apparatus 20. In this manner, data from the device can be correlated with a particular patient. Indeed, in some embodiments, patient support apparatus 20 is configured to automatically establish communication with the tagged device (via transceiver 124 and/or Bluetooth transceiver 122) and to forward data from that device to server 84, which in turn may automatically forward the data to the corresponding patient's electronic medical record. Because patient support apparatus 20 automatically determines that the device is positioned within the volume of space defined by association threshold 156, it is not necessary for a caregiver to take any manual steps to ensure that data from the tagged device is forwarded to the proper corresponding patient's electronic medical record because patient support apparatus 20, along with server 84, automatically determine the correct patient associated with that tagged device. Further details regarding at least one manner in which this automatic patient association may be made are found in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference.

It will be understood that a tag 140b may be attached to a wide variety of types of devices. Such devices include, but are not limited to, exercise devices, heel care boots, IV stands and/or poles, ventilators, patient monitors (e.g. saturated oxygen (Sp0₂) monitors), vital sign detectors (e.g. heart rate, breathing rate, temperature), patient positioning devices (e.g. wedges, turning devices, pumps), ambient sensors (e.g. air temperature, air flow, light, humidity, pressure, altitude, sound/noise), and/or any other types of devices that are used in the treatment, monitoring, and/or rehabilitation of the patient.

In at least one embodiment, patient support apparatus 20 may be configured to detect the position of an exercise device (and/or movement of the exercise device) by incorporating a tag 140b into the exercise device. One example of this is disclosed in commonly assigned U.S. patent application Ser. No. 63/161,175 filed Mar. 15, 2021, by inventors Krishna Bhimavarapu et al. and entitled EXERCISE DEVICE AND PATIENT SUPPORT APPARATUS, the complete disclosure of which is incorporated herein by reference.

In some embodiments, the transceivers 104, 124 may operate in the same manner as, and include any of the same functions as, the anchors and pseudo-anchors disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference.

In some embodiments, wall units 60 may also be utilized to determine the location of any of tags 140a and/or 140b, such as is disclosed in commonly assigned U.S. patent application Ser. No. 63/132,514 filed Dec. 31, 2020, by inventors Alexander Bodurka et al. and entitled PATIENT SUPPORT APPARATUS AND MEDICAL DEVICE NETWORKS, and in commonly assigned U.S. patent application Ser. No. 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosures of both of which are incorporated herein by reference.

As was mentioned previously, in some embodiments, tags 140a and 140b include different IDs, or different types of IDs, so that controller 132 is able to distinguish between tags 140a that are attached to a patient's body and tags 140b that are attached to devices. In some embodiments, the IDs may tell controller 132 additional information, such as the location of a particular tag 140a on the patient's body, the type of device to which a tag 140b is attached (e.g. a heel care boot, a vital signs monitor, a patient monitor, an IV stand, a therapy device, etc.), and/or other information. The ID of the tag 140b is transmitted to one or more of the transceivers 124 onboard patient support apparatus 20, and patient support apparatus 20 is configured to then forward the ID to server 84 and/or one or more electronic devices 96. The recipient of the ID has access to a data table that correlates the ID to a specific type of device, and the recipient can then share the fact that a particular type of device 166 is being used with a patient on a particular patient support apparatus 20. This sharing may take place via one or more of the electronic devices 96, thereby enabling the electronic devices 96 to display the type of device(s) being used with a particular patient. Data from the device may also be displayed on the same electronic device 96, thereby giving the viewer real time information about the devices being used with a particular patient support apparatus.

In any of the embodiments disclosed herein, server 84 may be configured to additionally execute a caregiver assistance software application of the type described in the following commonly assigned patent applications: U.S. patent application Ser. No. 62/826,097, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. That is, server 84 may be configured to share with one or more electronic devices any of the information shared with the electronic devices disclosed in these aforementioned patent applications. Thus, for example, server 84 may be configured to not only share the location of patient support apparatuses 20 (and any devices that may be associated with them) with electronic devices 96, but it may also forward data from patient monitoring system 136 to the electronic devices 96, thereby letting the caregivers associated with these devices know if, for example, the patient has exited patient support apparatus 20, is at risk of developing a bed sore, has not been turned in a given time period, and/or is experiencing a bout of restlessness. Alternatively, or additionally, patient support apparatus server 84 may forward patient support apparatus status data (e.g. current siderail position, bed exit status, brake status, height status, scale data, etc.) and/or caregiver rounding information (e.g. when the last rounding was performed for a particular patient, when the next rounds are due, etc.), and/or position data regarding any of the tags 140a, 140b that are positioned within range of UWB transceivers 124 to one or more electronic devices 96, thereby providing the caregivers associated with the devices 96 a consolidated portal (e.g. a single software application) for sharing this various information.

In some embodiments, wall units 60a and/or 60b may be configured to send a signal to patient support apparatus 20 (via UWB transceiver 104 and/or via BT transceiver 106) indicating what type of wall unit it is (e.g. whether it is a linked wall unit 60a or an unlinked wall unit 60b). Controller 132 of patient support apparatus 20 uses this information to determine whether or not to forward audio signals from microphone 138 to the wall unit 60 via BT transceiver 122 or UWB transceiver 124. More specifically, if the wall unit is a linked wall unit 60a, controller 132 forwards the audio signals (and linked wall unit 60a then forwards the audio signals to the communications outlet 64). If the wall unit is an unlinked wall unit 60b, controller 132 does not forward the audio signals (but instead forwards them to a nurse call cable port that receives a nurse call cable 66). The audio signals are generated from the patient's voice when he or she is talking to a remotely positioned caregiver via the healthcare facility's nurse call system.

It will also be understood that, although patient support apparatus 20 has been primarily described as having three UWB transceivers 134a-c (e.g. FIG. 8), patient support apparatus 20 may have different numbers of UWB transceivers in different embodiments, including embodiments with only a single transceiver 124, two UWB transceivers 124, four UWB transceivers 124, or more than four UWB transceivers 124.

Additionally, in some embodiments, patient support apparatus 20 may be adapted to display on one or more of its own displays—which may be included within any of control panels 54—its location as determined by its communication with one or more wall units 60. Additionally, or alternatively, patient support apparatus 20 may also be configured to display on display 52 the current position of any or all of the tags 140 whose current position it has determined.

It will also be understood that, in some embodiments, patient support apparatus 20 may include one or more UWB transceivers 124 that are positioned on movable components of patient support apparatus 20, such as, but not limited to, litter frame 28 and/or siderails 36. In such embodiments, controller 132 is apprised of the current location of each moveable transceiver 124 by way of one or more sensors that measure the current location of the one or moveable components. Controller 132 then uses this current location information when it communicates with one or more wall units 60 and/or with one or more of the tags 140.

It will be understood that, although the majority of the above-disclosure has discussed the use of transceivers 104 and 124 for determining the distances between themselves and/or one or more tags 140, transceivers 104 and/or 124 may be additionally and/or alternatively used for determining angular information between themselves and/or one or more tags. Thus, in some embodiments, transceivers 124 and the transceivers within tags 140 may be used to not only determine how far each transceiver 124 is positioned from the tag 140, but also the angular relationship of each transceiver 124 relative to the nearby tag 140.

It will also be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A patient support apparatus comprising:
a support surface adapted to support a patient;
a first transceiver coupled to a first location on the patient support apparatus; and
a controller adapted to use radio frequency (RF) communication between the first transceiver and a first tag to determine a first distance between the first tag and the first transceiver, wherein the first tag is coupled to a patient associated with the patient support apparatus.

2.-3. (canceled)

4. The patient support apparatus of claim 1 wherein the controller is further adapted to repetitively determine the first distance between the first tag and the first transceiver and to record changes in the first distance.

5.-49. (canceled)

50. A patient support apparatus comprising:
a support surface adapted to support a patient;
a first transceiver coupled to a first location on the patient support apparatus; and
a controller adapted to monitor movement of the patient using communications between a first tag attached to the patient and the first transceiver.

51. The patient support apparatus of claim 50 wherein the controller is further adapted to determine whether a second tag attached to a medical device should be associated with at least one of the patient or the patient support apparatus using communications between the second tag and the first transceiver.

52. The patient support apparatus of claim 51 further comprising:
a second transceiver coupled to a second location on the patient support apparatus;
a third transceiver coupled to a third location on the patient support apparatus; and
wherein the controller is also adapted to use communications between the first tag and the second and third transceivers to both monitor movement of the patient and to determine whether the second tag should be associated with the at least one of the patient or the patient support apparatus.

53. The patient support apparatus of claim 52 wherein the controller is further adapted to use radio frequency (RF) communications between the first tag and the first, second, and third transceivers to determine a first three dimensional position of the first tag, the controller further adapted to use radio frequency (RF) communications between the second tag and the first, second, and third transceivers to determine a second three dimensional position of the second tag.

54. The patient support apparatus of claim 53 further comprising a control panel including a display and an exit detection control adapted to arm an exit detection system;
wherein the controller is further adapted to receive a first identifier from the first tag and to allow the exit detection system to be armed only if both of the following conditions are met: (1) the three dimensional position of the first tag is inside a first volume of space, and (2) the first identifier indicates that the first tag is able to be used for monitoring patient exits.

55. The patient support apparatus of claim 53 wherein the first, second, and third transceivers are ultra-wideband transceivers.

56. The patient support apparatus of claim 55 wherein the controller is further adapted to determine if the second three dimensional position is located within a volume of space defined in a fixed relationship to the patient support apparatus.

57. The patient support apparatus of claim 56 wherein the controller is further adapted to automatically associate the second tag with the patient support apparatus if the second three dimensional position is inside the volume of space, and to not automatically associate the second tag with the patient support apparatus if the second three dimensional position is outside the volume of space.

58. The patient support apparatus of claim 57 further comprising a fourth transceiver adapted to communicate with server, and wherein the controller is adapted to send a message to the server indicating that the second tag is associated with the patient support apparatus if the second three dimensional position is inside the volume of space.

59. The patient support apparatus of claim 55 wherein the first, second, and third transceivers are further adapted to communicate with a wall unit affixed to a wall of a healthcare facility at a known location, and wherein the controller is adapted to determine a position of the patient support apparatus within the healthcare facility using communications between the wall unit and the first, second, and third transceivers.

60. The patient support apparatus of claim 52 wherein the controller is adapted to determine at least one of the following from monitoring movement of the patient on the support surface: (1) whether the patient has exited from the patient support apparatus; or (2) how frequently the patient moves while positioned on the support surface.

61. The patient support apparatus of claim 55 further comprising a control panel, the control panel including a patient monitoring control in communication with the controller, wherein when the user activates the patient monitoring control, the controller is adapted to monitor the first three dimensional position of the first tag and determine a cumulative amount of movement of the first tag.

62. The patient support apparatus of claim 61 further comprising a fourth transceiver adapted to communicate with server, and wherein the controller is adapted to send a message to the server if the cumulative amount of movement of the first tag over a first period of time does not exceed a first threshold.

63. The patient support apparatus of claim 62 wherein the first period of time and the first threshold are selected to assist in preventing bed sores from developing on the patient.

64. The patient support apparatus of claim 61 further comprising a fourth transceiver adapted to communicate with server, and wherein the controller is adapted to send a message to the server if the cumulative amount of movement of the first tag over a second period of time exceeds a second threshold.

65. The patient support apparatus of claim 64 wherein the second period of time and the second threshold are selected to assist in identifying patient restlessness.

66. The patient support apparatus of claim 53 wherein the controller is further adapted to use the first three dimensional position of the first tag to determine if the patient has been turned by a caregiver.

67. The patient support apparatus of claim 66 further comprising a fourth transceiver adapted to communicate with server, and wherein the controller is adapted to send a message to the server indicating if the patient has been turned by the caregiver.
